(19) United States
(12) Patent Application Publication
(10) Pub. No.: US 2007/0087345 A1
(43) Pub. Date: Apr. 19, 2007

(54) Assays for the direct measurement of gene dosage

(75) Inventors: Marilyn C. Olson-Munoz, Madison, WI (US); Michelle L. Curtis, Cottage Grove, WI (US); Kyle C. Armantrout, Los Angeles, CA (US); Feng Cao, Milwaukee, WI (US); Bonnie L Hurwitz, Madison, WI (US); Daniel K. Machmeier, Middleton, WI (US); Sara M. Olson, Cross Plains, WI (US); Hon S. Ip, Madison, WI (US); Robert W. Kwiatkowski Jr, Verona, WI (US); LuAnne Chehak, Janesville, WI (US)

Correspondence Address: MEDLEN & CARROLL, LLP, 101 HOWARD STREET, SUITE 350, SAN FRANCISCO, CA, 94105, US

(73) Assignee: THIRD WAVE TECHNOLOGIES, INC., Madison, WI (US)

(21) Appl. No.: 10/564,136

(22) PCT Filed: Jul. 9, 2004
(86) PCT No.: PCT/US04/22014

§ 371 Date: Dec. 18, 2006

Related U.S. Application Data
(60) Provisional application No. 60/486,273, filed on Jul. 10, 2003.
(60) Provisional application No. 60/535,747, filed on Jan. 12, 2004.

(51) Int. Cl. C12Q 1/68

(52) U.S. Cl. 435/6

(57) ABSTRACT

The present invention relates to compositions, methods, and kits for quantifying variations in gene copy number, e.g., of individual genes or of chromosomes or portions of chromosomes in an homogeneous reaction, without the need for target amplification, fragment size resolution, or microscopy.

### FIELD OF THE INVENTION

The present invention relates to compositions and methods for the detection and quantification of aneuploidy, and of variations in gene dosage. In particular, the present invention relates to compositions, methods, and kits for quantifying variations in gene dosage in a homogeneous reaction without the need for target amplification, fragment size resolution, or microscopy. Still more particularly, the present invention relates to compositions, methods and kits for using invasive cleavage structure assays (e.g., the INVADER assay) to screen nucleic acid samples, e.g., from patients, for the presence of variations in gene copy number, e.g., of individual genes or of chromosomes or portions of chromosomes. The present invention also relates to compositions, methods and kits for gene dosage in a single reaction container.

### BACKGROUND OF THE INVENTION

Variations in gene dosage are clinically significant indicators of disease states. Such variations arise due to errors in DNA replication and can occur in germ line cells, leading to congential defects and even embryonic demise, or in somatic cells, often resulting in cancer. These replication anomalies can cause deletion or duplication of parts of genes, full-length genes and their surrounding regulatory regions, megabase-long portions of chromosomes, or entire chromosomes.

Single-gene copy number abnormalities often play a role in cancer biology, typically by altering the level of expression of a key gene product, such as a tumor suppressor, transcription factor, or membrane receptor. Such increased or decreased expression can in turn affect cancer development, progression, and response to treatment. For example, amplification of the her 2/neu gene, which occurs in 20-30% of breast cancer cases and can range in magnitude from single copy to more than 20 copies per chromosome (described in U.S. Pat. No. 4,968,603), accelerates cancer progression and relapse, decreases survival time, and alters response to therapeutic treatments (Konigshoff, M. et al., Clinical Chemistry 49:2, 219-229 (2003)).

Chromosomal abnormalities affect gene dosage on a larger scale and can affect either the number or structure of chromosomes. Conditions wherein cells, tissues, or individuals have one or more whole chromosomes or segments of chromosomes either absent, or in addition to the normal euploid complement of chromosomes can be referred to as aneuploidy. Germline replication errors due to chromosome non-disjunction result in either monosomies (one copy of an autosomal chromosome instead of the usual two or only one sex chromosome) or trisomies (three copies). Such events, when they do not result in outright embryonic demise, typically lead to a broad array of disorders often recognized as syndromes, e.g., trisomy 21 and Down's syndrome, trisomy 18 and Edward's syndrome, and trisomy 13 and Patau's syndrome. Structural chromosome abnormalities affecting parts of chromosomes arise due to chromosome breakage, and result in deletions, inversions, translocations or duplications of large blocks of genetic material. These events are often as devastating as the gain or loss of the entire chromosome and can lead to such disorders as Prader-Willi syndrome (del 15q11-13), retinoblastoma (del 13q14), Cri du chat syndrome (del 5p), and others listed in U.S. Pat. No. 5,888,740, herein incorporated in its entirety by reference.

When chromosomal abnormalities arise in somatic cells, for example as the result of acquired mutations such as loss of heterozygosity (LOH) or gene duplication, they are often associated with cancer. For example, loss of all or part of chromosome 9 is associated with progression of bladder cancer (Tsukamoto, M. et al. Cancer Genetics and Cytogenetics 134, 41-45 (2002)). Chromosome abnormalities often accumulate throughout tumor development and are associated with progressively worse prognoses, for example, amplification of a region of chromosome 20 can be used as a prognostic indicator of breast cancer (described in U.S. Pat. No. 6,268,184).

A number of methods have been developed to detect variations in gene and chromosome copy number. Applications for such methods include prenatal screening, preimplantation genetic diagnosis (PGD), cancer screening, and tumor analysis. The first developed and still most widely used methods, generally classified as “cytogenic” methods involve microscopic visualization of chromosomes. The pioneering cytogenetic method is a technique for staining condensed chromosomes, termed “karyotyping,” and first described in the early 1960's (reviewed in McNeil, N. and Ried, T., Expert Reviews in Molecular Medicine 14: 1-14, September (2000)). The stained chromosomes are analyzed for overall shape, total number, variations of chromosomal regions and for anomalies (Seabright, M. Lancet: 1, 967 (1972), Caspersson, T., et al. Exp. Cell Res. 60: 315-319 (1970)). Karyotyping remains the gold standard and is often still the method of choice in cytogenetic laboratories. While such analysis can provide definitive evidence of trisomy, monosomy and some large-scale structural abnormalities such as loss of most or all of a chromosome arm, classic karyotyping nonetheless suffers from numerous limitations, particularly when applied in a clinical or diagnostic setting. Primary among these is limited resolution. The smallest changes typically discernable using classic karyotyping methods are on the order of 10 MB, i.e., roughly the width of a Giemsa-stained band. Furthermore, this type of analysis is not generally informative with regard to chromosomal translocations (Tyessier, J. R. Cancer Genet. Cytogenet. 37:103 (1989). In addition, traditional stain-based karyotyping is limited to certain applications relates to the types of samples suitable for analysis. Typically, large numbers of living, dividing cells, e.g., in culture, are required; the approach is thus not suitable for archived samples. Finally, conventional karyotype analysis is time consuming, labor intensive, and requires a high degree of skill.

Molecular cytogenetic techniques are distinguished from classic karyotyping by their reliance on nucleic acid hybridization, in lieu of pure chemical staining, to visualize select chromosomal regions. Among the first such methods developed was Comparative Genomic Hybridization, or CGH, (described in U.S. Pat. No. 6,159,685 and related applications, herein incorporated in their entirety by reference). In CGH, genomic DNA is isolated from one or more test samples (e.g., tumor cells, embryos) and from a reference sample (e.g., a healthy cell). Each DNA preparation is labeled with a distinguishable label, such as fluorescent dyes having different absorption/emission spectra. By comparing different ratios across or within given chromosomes, this method can be used to compare copy numbers of different sequences within a single sample or between samples. A key advantage of CGH relative to classic karyotyping is its suitability for using archived, formalin-fixed paraffin-embedded specimens (Struski, S. et al., Cancer Genetics and Cytogenetics 135: 63-90 (2002)). However, as with conventional karyotyping, while accurate for analyzing chromosomal copy number abnormalities, CGH has limited resolution of deletions and amplifications, on the order of 3-20 Mb (Struski, S., supra and Lichter, P. J. Mol. Diagn. 2: 171-173 (2000)).

An alternative molecular cytogenetic method with enhanced resolution relative to CGH is fluorescence in situ hybridization, or FISH, in which nucleic acid probes, often several kb in length, are labeled with fluorophores and hybridized to isolated chromosomes, (described in U.S. Pat. Nos. 5,663,319, 6,300,066 and related applications and reviewed in McNeil and Ried, supra and Tepperberg, J. et al. Prenat. Diagn. 21: 293-301 (2001)). In some cases, efforts are made to select probes that are specific for individual chromosomes. The resulting hybrids are viewed through a microscope and analyzed for the extent and location of fluorescent signal. A related method is chromosome painting, described in U.S. Pat. Nos. 6,255,465, 6,270,971 and related patents, herein incorporated by reference. This method involves hybridizing to a genomic DNA sample a multiplicity of different labeled chromosome-specific probes, prepared by isolating chromosomes, usually by flow cytometry. Individual chromosomes are then visualized by fluorescence microscopy.

Another method involves comparing sample chromosomal DNA to a reference based on the presence or absence of restriction fragment length polymorphisms, either by restriction endonuclease digestion or PCR amplification, followed in each case by hybridization to labeled probes comprising the polymorphic site, as described in U.S. Pat. Nos. 5,380,645, 5,580,729 and related applications).

Efforts to develop still more rapid and higher throughput methods for analyzing aberrations in chromosome copy number and gene dosage have led to the development of PCR-based approaches. Various quantitative PCR strategies have been applied to the determination of copy number, including real time fluorescence PCR (e.g., that described in U.S. Pat. No. 6,180,349), quantitative fluorescence PCR (QF-PCR) (e.g., Bili, C. et al. Prenat. Diagn. 22: 360-365 (2002)), quantitative PCR (Q-PCR) using internal controls selected to match the target sequence in length and GC content (e.g., U.S. Pat. No. 5,888,740). QF-PCR methods have been developed for detecting aneuploidy of chromosomes 13, 18, 21, X and Y using highly polymorphic, chromosome-specific short tandem repeats (STRs) as chromosome-specific markers (described in Findlay, I. et al. J. Assist. Reprod. Genet. 15: 266-75 (1998); Cirigliano, V. et al., Ann. Hum. Genet. 65: 421-427 (2001) and Cirigliano, V. et al. Prenat. Diagn. 19: 1099-1103 (1999)). Such methods have also been used to test for selected translocations (Adinolfi, M. and Sherlock, J., Lancet 358: 1030-1 (2001)) and for deletions, duplications and gene dosage (Ruiz-Ponte, C. et al. Clinical Chem. 46:1574-1582 (2000); Poropat, R. A. and Nicholson, G. A., Clinical Chem. 44: 724-730 (1988); and Konigshoff, M. et al., supra.)

Other PCR-based methods target non-repetitive, gene-based sequences. Rahil et al. described a QF-PCR method directed to various genic regions on chromosomes 13, 18, and 21 Rahil, H. et al., European J Hum Gen 10:462-466 (2002). An alternative approach, termed Multiplex Ligation-dependent Probe Amplification (MLPA) involves ligation of chromosome-specific probes comprising distinct “tails”, which are subsequently PCR-amplified to yield fragments of specific length indicative of the presence of the target being detected. These amplified fragments are then separated by size to indicate which of the probed chromosomal regions are present, absent, or duplicated (Schouten, J. P., et al., Nucleic Acids Res. 30: e 57 (2002)). In general, such PCR-based methods have the advantage of being applicable to a variety of biological sample types, including blood, cultured amniocytes, amniotic fluid, urine, etc. They are also more amenable to high throughput analysis and execution by machines or technicians than are cytogenetic methods requiring microscopic analysis. Results obtained using such methods are often available in a matter of hours or days. However, it is typically necessary to analyze multiple loci per chromosome with such approaches, since any homozygosity or preferential amplification of only one allele of a locus may occur, affecting interpretation of the results (Adinolfi, M. and Sherlock, J., ibid). Moreover, because of the dangers of false positive reactions, these PCR-based procedures require rigid controls to prevent contamination and carry over (Ehrlich et al., in PCR-Based Diagnostics in Infectious Diseases, Ehrlich and Greenberg (eds), Blackwell Scientific Publications, [1994], pp. 3-18).

Therefore, there exists a need for a rapid and quantitative detection assay for measuring aneuploidy and gene dosage directly, without the need without the need for target amplification, fragment size resolution, or microscopy.

### SUMMARY OF THE INVENTION

The present invention provides compositions and methods for the detection and characterization of mutations resulting in alterations in gene dosage. More particularly, the present invention provides compositions, methods and kits for using invasive cleavage structure assays (e.g., the INVADER assay) to screen nucleic acid samples, e.g., from patients, for the presence of variations resulting in changes in gene copy number. The present invention also provides compositions, methods and kits for screening patient samples in a single reaction container.

In some embodiments, the present invention provides a method for selecting a chromosome-specific oligonucleotide sequence, comprising identifying a chromosome-specific genic sequence that is unique in a genome, identifying an exon tag sequence within the genic sequence, wherein the exon tag sequence is compared to the genome to determine that the exon tag sequence is unique within the genome, and selecting an oligonucleotide sequence complementary to the exon tag or its complement. In some preferred embodiments, the exon tag sequence within the genic sequence is less than 100 base pairs in length. In some particularly preferred embodiments, the exon tag sequence within the genic sequence is 91 base pairs in length. In other preferred embodiments, the exon tag sequence is the length of an entire exon.

In some embodiments, the selection of an oligonucleotide sequence complementary to the exon tag or its complement comprises selecting an oligonucleotide sequence having 20% to 70%, and preferably 40-60% GC content.

Some embodiments of the present invention provide a method for detecting aneuploidy of a chromosome in a subject, comprising the steps of: a) selecting an exon tag sequence for the chromosome; b) providing a non-amplifying oligonucleotide detection assay configured to detect the exon tag sequence or its complement; and c) detecting the exon tag with the non-amplifying oligonucleotide detection assay.

In some embodiments, the selecting of an exon tag sequence comprises the steps of: a) identifying a genic sequence that is specific to the chromosome in the subject, and that is unique in the genome of the species of the subject; and b) identifying an exon tag sequence within the genic sequence, wherein the exon tag sequence is compared to the genome to determine that the exon tag sequence is unique within the genome of the species of the subject.

In some embodiments, the method of the present invention further comprises providing an internal control and a non-amplifying oligonucleotide detection assay configured to detect the internal control, wherein the internal control target is detected using the non-amplifying oligonucleotide detection assay configured to detect the internal control.

In some embodiments the present invention provides a method for detecting aneuploidy of a chromosome in a subject, comprising the steps of: a selecting an exon tag sequence for the chromosome; b) providing a non-amplified oligonucleotide detection assay configured to detect the exon tag sequence or its complement; and c) detecting the exon tag with the non-amplified oligonucleotide detection assay.

In some preferred embodiments, the selecting of an exon tag sequence comprises the steps of: a) identifying a genic sequence that is specific to the chromosome in the subject, and that is unique in the genome of the species of the subject; b) identifying an exon tag sequence within the genic sequence, wherein the exon tag sequence is compared to the genome to determine that the exon tag sequence is unique within the genome of the species of the subject.

In some preferred embodiments, the method further comprises providing an internal control and a non-amplifying oligonucleotide detection assay configured to detect the internal control, wherein the internal control target is detected using the non-amplifying oligonucleotide detection assay configured to detect the internal control. In some particularly preferred embodiments, the internal control comprises a sequence from a gene on chromosome 1.

In some embodiments of the methods of the present invention, the chromosome in a subject is selected from the group consisting of chromosomes 13, 18, 21, X and Y. In some embodiments of the methods of the present invention, the exon tag sequence is contained in a sample type including, but not limited to amniocyte cells or cell culture, amniotic fluid, placental tissue (villi) obtained by CVS techniques, or other tissues/cells of embryonic origin (e.g., including, but not limited to, cystic hygroma fluid, fetal urine, fetal skin, and fetal blood). In some embodiments of methods of the present invention, DNA (is) isolated from maternal plasma or serum and fetal DNA present in the sample is assayed.

In some embodiments of methods of the present invention, DNA (is) isolated from maternal plasma or serum and fetal DNA present in the sample is assayed. The present invention provides kits for the determination of aneuploidy. In some embodiments, kits of the present invention comprise a non-amplified oligonucleotide detection assay configured for detecting at least one exon tag. In some embodiments of kits, the non-amplified oligonucleotide detection assay comprises first and second oligonucleotides configured to form an invasive cleavage structure in combination with a target sequence comprising the at least one exon tag. In some preferred embodiments, the first oligonucleotide comprises a 5′ portion and a 3′ portion, wherein the 3′ portion is configured to hybridize to the target sequence, and wherein the 5′ portion is configured to not hybridize to the target sequence. In some particularly preferred embodiments, the second oligonucleotide comprises a 5′ portion and a 3′ portion, wherein the 5′ portion is configured to hybridize to the target sequence, and wherein the 3′ terminal nucleotide is configured to hybridize or not hybridize to the target sequence.

In some preferred embodiments, the kit of the present invention is configured to detect an exon tag from a gene on a targeted chromosome (13, 18, 21, X and Y) including, but not limited to, DSCR9, DLEU1, FLJ23403, PFKFB1, NRIP1, SRY, PCDH9, CN2, PRKY, HLCS, MTMR8, FLJ21174, or PCTK1. In still other embodiments, the kit of the present invention comprises an internal control. In particularly preferred embodiments, the internal control comprises a sequence from genes on chromosome 1 (e.g., ACTA1 and HIST2HBE).

Definitions

To facilitate an understanding of the present invention, a number of terms and phrases are defined below:

The term “gene dosage” as used herein refers to the copy number of a gene, a genic region, a chromosome, or fragments or portions thereof. Normal individuals carry two copies of most genes or genic regions, one on each of two chromosomes. However, there are certain exceptions, e.g., when genes or genic regions reside on the X or Y chromosomes, or when genes sequences are present in pseudogenes.

The term “aneuploidy” as used herein refers to conditions wherein cells, tissues, or individuals have one or more whole chromosomes or segments of chromosomes either absent, or in addition to the normal euploid complement of chromosomes.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “genic region” as used herein refers to a gene, its exons, its introns, and its regions flanking it upstream and downstream, e.g., 5 to 10 kilobases 5′ and 3′ of the transcription start and stop sites, respectively.

The term “genic sequence” as used herein refers to the sequence of a gene, its introns, and its regions flanking it upstream and downstream, e.g., 5 to 10 kilobases 5′ and 3′ of the transcription start and stop sites, respectively.

The term “chromosome-specific” as used herein refers to a sequence that is found only in that particular type of chromosome.

The term “exon tag” as used herein refers to a chromosome-specific sequence in the exon of a gene that is also unique in the genome.

As used herein, the terms “subject” and “patient” refer to any organisms including plants, microorganisms and animals (e.g., mammals such as dogs, cats, livestock, and humans).

As used herein, the term “INVADER assay reagents” refers to one or more reagents for detecting target sequences, said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In some embodiments, the oligonucleotides comprise first and second oligonucleotides, said first oligonucleotide comprising a 5′ portion complementary to a first region of the target nucleic acid and said second oligonucleotide comprising a 3′ portion and a 5′ portion, said 5′ portion complementary to a second region of the target nucleic acid downstream of and contiguous to the first portion. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid.

In some embodiments, INVADER assay reagents are configured to detect a target nucleic acid sequence comprising first and second non-contiguous single-stranded regions separated by an intervening region comprising a double-stranded region. In preferred embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components”.

In some embodiments, the INVADER assay reagents further comprise a third oligonucleotide complementary to a third portion of the target nucleic acid upstream of the first portion of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In still other embodiments, the INVADER assay reagents further comprise an ARRESTOR molecule (e.g., ARRESTOR oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In preferred embodiments, predispensed assay reagent components are predispensed and are provided in a reaction vessel (including but not limited to a reaction tube or a well, as in, e.g., a microtiter plate). In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but is not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety positive or negative charge) or alternatively, may be charge neutral. Labels can include or consist of nucleic acid or protein sequence, so long as the sequence comprising the label is detectable.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the terms “complementary” or “complementarity” are used in reference to polynucleotides (i.e., a sequence of nucleotides such as an oligonucleotide or a target nucleic acid) related by base-pairing rules. For natural bases, the base pairing rules are generally those developed by Watson and Crick. For example, for the sequence “5′-A-G-T-3′,” is complementary to the sequence “3′-T-C-A-5′.” For non-natural bases, base-pairing rules include the formation of hydrogen bonds in a manner similar to the Watson-Crick base pairing rules (for example, having similar features such as geometries or bond angles, as described, e.g., by Kunkel, et al., Annu Rev Biochem. 69:497-529 (2000), incorporated herein by reference). Complementarity may be “partial,” in which only some of the nucleic acids' bases are matched according to base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand. Nucleotide analogs used to form non-standard base pairs, whether with another nucleotide analog (e.g., an IsoC/IsoG base pair), or with a naturally occurring nucleotide (e.g., as described in U.S. Pat. No. 5,912,340, herein incorporated by reference in its entirety) are also considered to be complementary to a base pairing partner within the meaning this definition. Further, when nucleotides are known to form pairs with multiple different bases, e.g., the IsoG nucleotide's ability to pair with IsoC and with T nucleotides, each of the bases with which it can form a hydrogen-bonded base-pair falls within the meaning of “complementary,” as used herein. “Universal” bases, ie., those that can form base pairs with several other bases, such as the “wobble” base inosine, are considered complementary to those bases with which pairs can be formed.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (ie., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value May be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tₘ.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “recombinant DNA vector” as used herein refers to DNA sequences containing a desired heterologous sequence. For example, although the term is not limited to the use of expressed sequences or sequences that encode an expression product, in some embodiments, the heterologous sequence is a coding sequence and appropriate DNA sequences necessary for either the replication of the coding sequence in a host organism, or the expression of the operably linked coding sequence in a particular host organism. DNA sequences necessary for expression in prokaryotes include a promoter, optionally an operator sequence, a ribosome binding site and possibly other sequences. Eukaryotic cells are known to utilize promoters, polyadenlyation signals and enhancers.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5“end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, may be made using molecular biological methods, e.g., purification of a restriction digest, or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “cleavage structure” as used herein, refers to a structure that is formed by the interaction of at least one probe oligonucleotide and a target nucleic acid, forming a structure comprising a duplex, the resulting structure being cleavable by a cleavage means, including but not limited to an enzyme. The cleavage structure is a substrate for specific cleavage by the cleavage means in contrast to a nucleic acid molecule that is a substrate for non-specific cleavage by agents such as phosphodiesterases which cleave nucleic acid molecules without regard to secondary structure (i.e., no formation of a duplexed structure is required).

The term “cleavage means” or “cleavage agent” as used herein refers to any means that is capable of cleaving a cleavage structure, including but not limited to enzymes. “Structure-specific nucleases” or “structure-specific enzymes” are enzymes that recognize specific secondary structures in a nucleic molecule and cleave these structures. The cleavage means of the invention cleave a nucleic acid molecule in response to the formation of cleavage structures; it is not necessary that the cleavage means cleave the cleavage structure at any particular location within the cleavage structure.

The cleavage means may include nuclease activity provided from a variety of sources including the Cleavase enzymes, the FEN-1 endonucleases (including RAD2 and XPG proteins), Taq DNA polymerase and E. coli DNA polymerase I. The cleavage means may include enzymes having 5′ nuclease activity (e.g., Taq DNA polymerase (DNAP), E. coli DNA polymerase I). The cleavage means may also include modified DNA polymerases having 5′ nuclease activity but lacking synthetic activity. Examples of cleavage means suitable for use in the method and kits of the present invention are provided in U.S. Pat. Nos. 5,614,402; 5,795,763; 5,843,669; 6,090,606, 6,562,611, 6553,587; PCT Appln. Nos WO 98/23774; WO 02/070755; and WO01/90337, each of which is herein incorporated by reference it its entirety.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage means with a cleavage structure (i.e., the treatment of a cleavage structure with a cleavage means).

The term “target nucleic acid” refers to a nucleic acid molecule containing a sequence that has at least partial complementarity with at least a probe oligonucleotide and may also have at least partial complementarity with an INVADER oligonucleotide. The target nucleic acid may comprise single- or double-stranded DNA or RNA.

The term “non-target cleavage product” refers to a product of a cleavage reaction that is not derived from the target nucleic acid. As discussed above, in the methods of the present invention, cleavage of the cleavage structure generally occurs within the probe oligonucleotide. The fragments of the probe oligonucleotide generated by this target nucleic acid-dependent cleavage are “non-target cleavage products.”

The term “probe oligonucleotide” refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide—whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “cassette” as used herein refers to an oligonucleotide or combination of oligonucleotides configured to generate a detectable signal in response to cleavage of a probe oligonucleotide in an INVADER assay. In preferred embodiments, the cassette hybridizes to a non-target cleavage product from cleavage of the probe oligonucleotide to form a second invasive cleavage structure, such that the cassette can then be cleaved.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label. In particularly preferred embodiments, cassette comprises labeled moieties that produce a fluorescence resonance energy transfer (FRET) effect.

The term “substantially single-stranded” when used in reference to a nucleic acid substrate means that the substrate molecule exists primarily as a single strand of nucleic acid in contrast to a double-stranded substrate which exists as two strands of nucleic acid which are held together by inter-strand base pairing interactions.

As used herein, the phrase “non-amplified oligonucleotide detection assay” refers to a detection assay configured to detect the presence or absence of a particular polymorphism (e.g., SNP, repeat sequence, etc.) in a target sequence (e.g., genomic DNA) that has not been amplified (e.g., by PCR), without creating copies of the target sequence. A “non-amplified oligonucloetide detection assay” may, for example, amplify a signal used to indicate the presence or absence of a particular polymorphism in a target sequence, so long as the target sequence is not copied.

As used herein, the phrase “non-amplifying oligonucleotide detection assay” refers to a detection assay configured to detect the presence or absence of a particular polymorphism (e.g., SNP, repeat sequence, etc.) in a target sequence (e.g., genomic DNA, or amplified or other synthetic DNA), without creating copies of the target sequence. A “non-amplifying oligonucloetide detection assay” may, for example, amplify a signal used to indicate the presence or absence of a particular polymorphism in a target sequence, so long as the target sequence is not copied.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “liberating” as used herein refers to the release of a nucleic acid fragment from a larger nucleic acid fragment, such as an oligonucleotide, by the action of, for example, a 5′ nuclease such that the released fragment is no longer covalently attached to the remainder of the oligonucleotide.

The term “Kₘ” as used herein refers to the Michaelis-Menten constant for an enzyme and is defined as the concentration of the specific substrate at which a given enzyme yields one-half its maximum velocity in an enzyme catalyzed reaction.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include base analogs, and comprise modified forms of deoxyribonucleotides as well as ribonucleotides, and include but are not limited to modified bases and nucleotides described in U.S. Pat. Nos. 5,432,272; 6,001,983; 6,037,120; 6,140,496; 5,912,340; 6,127,121 and 6,143,877, each of which is incorporated herein by reference in their entireties; heterocyclic base analogs based on the purine or pyrimidine ring systems, and other heterocyclic bases.

The term “polymorphic locus” is a locus present in a population that shows variation between members of the population (e.g., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

The term “microorganism” as used herein means an organism too small to be observed with the unaided eye and includes, but is not limited to bacteria, virus, protozoans, fungi, and ciliates.

The term “microbial gene sequences” refers to gene sequences derived from a microorganism.

The term “bacteria” refers to any bacterial species including eubacterial and archaebacterial species.

The term “virus” refers to obligate, ultramicroscopic, intracellular parasites incapable of autonomous replication (i.e., replication requires the use of the host cell's machinery).

The term “multi-drug resistant” or multiple-drug resistant” refers to a microorganism that is resistant to more than one of the antibiotics or antimicrobial agents used in the treatment of said microorganism.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The term “source of target nucleic acid” refers to any sample that contains nucleic acids (RNA or DNA). Particularly preferred sources of target nucleic acids are biological samples including, but not limited to blood, saliva, cerebral spinal fluid, pleural fluid, milk, lymph, sputum and semen.

An oligonucleotide is said to be present in “excess” relative to another oligonucleotide (or target nucleic acid sequence) if that oligonucleotide is present at a higher molar concentration that the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present at least a 100-fold molar excess; typically at least 1 pmole of each probe oligonucleotide would be used when the target nucleic acid sequence was present at about 10 fmoles or less.

A sample “suspected of containing” a first and a second target nucleic acid may contain either, both or neither target nucleic acid molecule.

The term “reactant” is used herein in its broadest sense. The reactant can comprise, for example, an enzymatic reactant, a chemical reactant or light (e.g., ultraviolet light, particularly short wavelength ultraviolet light is known to break oligonucleotide chains). Any agent capable of reacting with an oligonucleotide to either shorten (i.e., cleave) or elongate the oligonucleotide is encompassed within the term “reactant.”

As used herein, the term “purified” or “to purify” refers to the removal of contaminants from a sample. For example, recombinant CLEAVASE nucleases are expressed in bacterial host cells and the nucleases are purified by the removal of host cell proteins; the percent of these recombinant nucleases is thereby increased in the sample.

As used herein the term “portion” when in reference to a protein (as in “a portion of a given protein”) refers to fragments of that protein. The fragments may range in size from four amino acid residues to the entire amino acid sequence minus one amino acid (e.g., 4, 5, 6, . . . , n−1).

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

The term “continuous strand of nucleic acid” as used herein is means a strand of nucleic acid that has a continuous, covalently linked, backbone structure, without nicks or other disruptions. The disposition of the base portion of each nucleotide, whether base-paired, single-stranded or mismatched, is not an element in the definition of a continuous strand. The backbone of the continuous strand is not limited to the ribose-phosphate or deoxyribose-phosphate compositions that are found in naturally occurring, unmodified nucleic acids. A nucleic acid of the present invention may comprise modifications in the structure of the backbone, including but not limited to phosphorothioate residues, phosphonate residues, 2′ substituted ribose residues (e.g., 2′-O-methyl ribose) and alternative sugar (e.g., arabinose) containing residues.

The term “continuous duplex” as used herein refers to a region of double stranded nucleic acid in which there is no disruption in the progression of basepairs within the duplex (i.e., the base pairs along the duplex are not distorted to accommodate a gap, bulge or mismatch with the confines of the region of continuous duplex). As used herein the term refers only to the arrangement of the basepairs within the duplex, without implication of continuity in the backbone portion of the nucleic acid strand. Duplex nucleic acids with uninterrupted basepairing, but with nicks in one or both strands are within the definition of a continuous duplex.

The term “duplex” refers to the state of nucleic acids in which the base portions of the nucleotides on one strand are bound through hydrogen bonding the their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

The term “template” refers to a strand of nucleic acid on which a complementary copy is built from nucleoside triphosphates through the activity of a template-dependent nucleic acid polymerase. Within a duplex the template strand is, by convention, depicted and described as the “bottom” strand. Similarly, the non-template strand is often depicted and described as the “top” strand.

### DESCRIPTION OF THE DRAWINGS

The following figures form part of the present specification and are included to further demonstrate certain aspects and embodiments of the present invention.

FIG. 1 shows a general overview of the biplex INVADER assay.

FIG. 2 shows a general overview of the EXON TAGGER program.

FIG. 3 shows a list of design regions and oligonucleotide designs for INVADER assays for aneuploidy. All oligonucleotide sequences are shown in the 5′-3′ orientation and all probes contain 3′ hexanediol. GC content refers to the GC content of the target 91 mer.

FIG. 4 illustrates determination of the limit of detection (LOD) of assays to detect chromosome 21.

FIG. 5 illustrates detection of discrete regions of chromosome 13.

FIG. 6 illustrates detection of discrete regions of chromosome 18.

FIG. 7A-F illustrates detection of discrete regions of chromosome 21.

FIG. 8 illustrates detection of discrete regions of the X chromosome.

FIG. 9 illustrates detection of discrete regions of the Y chromosome.

FIG. 10 shows a comparison of detection of the DSCR6 gene by two different probe sets.

FIG. 11 shows the results of an experiment to mimic detection of a trisomy 21 sample contaminated with varying levels of normal, disomy DNA.

FIG. 12 shows a list of target regions for INVADER assays for aneuploidy. The INVADER assay footprint is indicated in parenthesis and the cleavage site is designated in brackets.

FIG. 13 illustrates determination of the limit of detection (LOD) of assays to detect the X chromosome. FIG. 13A shows detection of PFKFB1+PCTK1 targets and FIG. 13B shows detection of MTMR8+FLJ21174 targets.

FIG. 14 shows the results of the INVADER assay for detection of chromosome 18 targets in samples of mixed content.

FIG. 15 shows the results of INVADER assay detection of triploidy samples.

### DETAILED DESCRIPTION OF THE INVENTION

The present invention provides methods for measuring the copy number of a specific polynucleotide sequence in a biological sample. In general, the present invention involves homogeneous detection of gene dosage in a single reaction vessel. In some embodiments, gene dosage is measured in a single reaction vessel. In some embodiments, accumulation of target-specific signal is directly correlated to the amount of the specific polynucleotide sequence present in the sample.

In some embodiments, the methods of the present invention involve direct detection of a test polynucleotide sequence and of a control sequence. In some embodiments, the number of copies of a normal individual will be two, and gene dosage determinations that deviate from two will be deemed aberrant.

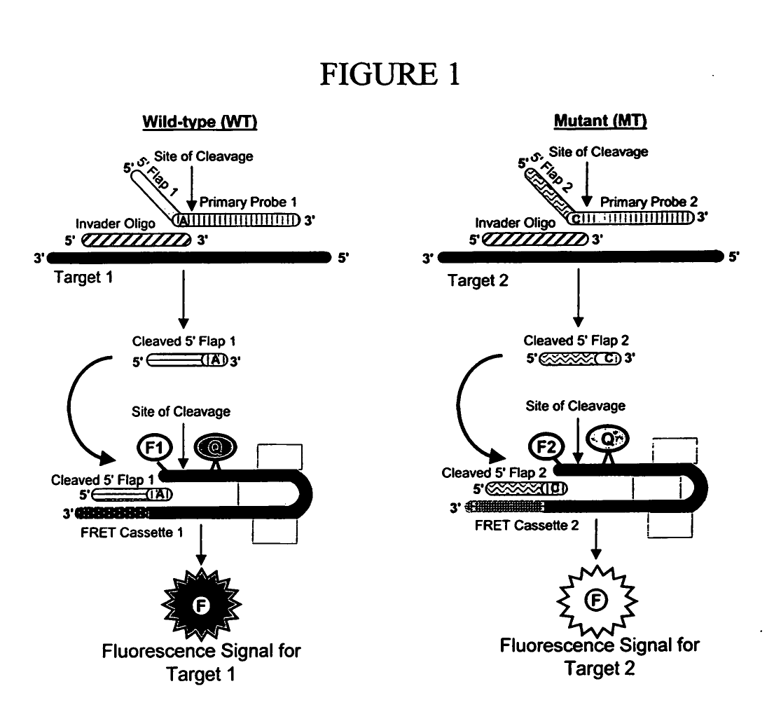

In some preferred embodiments, the methods of the present invention involve the INVADER assay. The INVADER assay detects specific DNA and RNA sequences by using structure-specific enzymes (e.g., FEN endonucleases) to cleave a complex formed by the hybridization of overlapping oligonucleotide probes (See, e.g., FIG. 1). When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced. Such embodiments have been termed the INVADER assay (Third Wave Technologies) and are described in U.S. Pat. Nos. 5,846,717, 5,985,557, 5,994,069, 6,001,567, and 6,090,543, WO 97/27214 WO 98/42873, Lyamichev et al., Nat. Biotech., 17:292 (1999), Hall et al., PNAS, USA, 97:8272 (2000), each of which is herein incorporated by reference in their entirety for all purposes). In preferred embodiments, elevated temperature and an excess of one of the probes enable multiple probes to be cleaved for each target sequence present without temperature cycling. The resulting cleavage products are indicative of the presence of specific target nucleic acid sequences in the sample. The reactions can be configured such that the amount of the cleavage product produced indicates the amount of the target sequence present in the reaction. Lyamichev, et al, Nature Biotech 1999, supra.

The INVADER assay detects hybridization of probes to a target by enzymatic cleavage of specific structures by structure specific enzymes (See, INVADER assays, Third Wave Technologies; See e.g., U.S. Pat. Nos. 5,846,717; 6,090,543; 6,001,567; 5,985,557; 6,090,543; 5,994,069; Lyamichev et al., Nat. Biotech., 17:292 (1999), Hall et al., PNAS, USA, 97:8272 (2000), WO97/27214 and WO98/42873, each of which is herein incorporated by reference in their entirety for all purposes).

The INVADER assay can be configured to detect gene dosage or specific mutations and SNPs in unamplified, as well as amplified, RNA and DNA including genomic DNA. In the embodiments shown schematically in FIG. 1, the INVADER assay uses two cascading steps (e.g., a primary and a secondary reaction) to generate and then to amplify the target-specific signal. For convenience, the targets in the following discussion are described as Target 1 and Target 2, even though this terminology does not apply to all genetic variations. For example, in some embodiments, Target 1 is a wild type form of a gene and Target 2 is a variant or mutant form of a gene. In other embodiments, Target 1 and Target 2 are genes present in different copy numbers in different samples. In the primary reaction (FIG. 1), the Target 1 primary probe and the INVADER oligonucleotide hybridize in tandem to the target nucleic acid to form an overlapping structure. An unpaired “flap” is included on the 5′ end of the Target 1 primary probe. A structure-specific enzyme (e.g., a CLEAVASE enzyme, Third Wave Technologies) recognizes the overlap and cleaves off the unpaired flap, releasing it as a target-specific product. In the secondary reaction, this cleaved product serves as an INVADER oligonucleotide on the Target 1 fluorescence resonance energy transfer (WT-FRET) probe to again create the structure recognized by the structure specific enzyme (panel A). When the two dyes on a single FRET probe are separated by cleavage (indicated by the arrow in FIG. 1), a detectable fluorescent signal above background fluorescence is produced. Consequently, cleavage of this second structure results in an increase in fluorescence, indicating the presence of the Target 1 allele. In some embodiments, FRET probes having different labels (e.g., resolvable by difference in emission or excitation wavelengths, or resolvable by time-resolved fluorescence detection) are provided for each allele or locus to be detected, such that the different alleles or loci can be detected in a single reaction. In such embodiments, the primary probe sets and the different FRET probes may be combined in a single assay, allowing comparison of the signals from each allele or locus in the same sample.

FIG. 1 shows one illustrative embodiments of the INVADER assay where wild type and mutant alleles are detected. In the embodiment shown in FIG. 1, the primary INVADER assay reaction is directed against the target DNA (or RNA) being detected. Generally, the target DNA is the limiting component in the first invasive cleavage, since the INVADER and primary probe are generally supplied in molar excess. In the second invasive cleavage, it is the released flap that is limiting. When these two cleavage reactions are performed sequentially, the fluorescence signal from the composite reaction accumulates exponentially, and quantitatively reflects the amount of target DNA amount. Hall, et al., PNAS 2000, supra.

In the embodiment shown in FIG. 1, if the primary probe oligonucleotide and the target nucleotide sequence do not match (complement) at the cleavage site (e.g., as with the MT primary probe and the WT target, FIG. 1, panel B), the overlapped structure does not form and cleavage is suppressed. The structure-specific enzyme (e.g., CLEAVASE VIII enzyme, Third Wave Technologies) cleaves the overlapped structure more efficiently (e.g., at least 340-fold) than the non-overlapping structure, allowing excellent discrimination of the alleles.

The assay is generally configured such that the probes turn over without temperature cycling to produce many signals per target (i.e., linear signal amplification). Similarly, each target-specific product can enable the cleavage of many FRET probes. In alternative embodiments, the assay can be configured to use temperature cycling to facilitate probe turnover.

In certain embodiments, the INVADER assay, or other nucleotide detection assays, are performed with oligonucleotides selected to detect sites on a target strand that have been found to be particularly accessible. In some embodiments, the INVADER assays are performed using one or more structure bridging oligonucleotides. Such methods, procedures and compositions are described in U.S. Pat. No. 6,194,149, WO9850403, and WO198537, all of which are specifically incorporated by reference in their entireties.

In certain embodiments, the target nucleic acid sequence is amplified prior to detection (e.g., such that synthetic nucleic acid is generated). In some embodiments, the target nucleic acid comprises genomic DNA. In other embodiments, the target nucleic acid comprises synthetic DNA or RNA. In some preferred embodiments, synthetic DNA within a sample is created using a purified polymerase. In some preferred embodiments, creation of synthetic DNA using a purified polymerase comprises the use of PCR. In other preferred embodiments, creation of synthetic DNA using a purified DNA polymerase, suitable for use with the methods of the present invention, comprises use of rolling circle amplification, (e.g., as in U.S. Pat. Nos. 6,210,884, 6,183,960 and 6,235,502, herein incorporated by reference in their entireties). In other preferred embodiments, creation of synthetic DNA comprises copying genomic DNA by priming from a plurality of sites on a genomic DNA sample. In some embodiments, priming from a plurality of sites on a genomic DNA sample comprises using short (e.g., fewer than about 8 nucleotides) oligonucleotide primers. In other embodiments, priming from a plurality of sites on a genomic DNA comprises extension of 3′ ends in nicked, double-stranded genomic DNA (ie., where a 3′ hydroxyl group has been made available for extension by breakage or cleavage of one strand of a double stranded region of DNA). Some examples of making synthetic DNA using a purified polymerase on nicked genomic DNAs, suitable for use with the methods and compositions of the present invention, are provided in U.S. Pat. No. 6,117,634, issued Sep. 12, 2000, and U.S. Pat. No. 6,197,557, issued Mar. 6, 2001, and in PCT application WO 98/39485, each incorporated by reference herein in their entireties for all purposes.

In other embodiments, synthetic DNA suitable for use with the methods and compositions of the present invention is made using a purified polymerase on multiply-primed genomic DNA, as provided, e.g., in U.S. Pat. Nos. 6,291,187, and 6,323,009, and in PCT applications WO 01/88190 and WO 02/00934, each herein incorporated by reference in their entireties for all purposes. In these embodiments, amplification of DNA such as genomic DNA is accomplished using a DNA polymerase, such as the highly processive φ 29 polymerase (as described, e.g., in U.S. Pat. Nos. 5,198,543 and 5,001,050, each herein incorporated by reference in their entireties for all purposes) in combination with exonuclease-resistant random primers, such as hexamers.

In certain embodiments, the present invention provides kits for assaying a pooled sample (e.g., a pooled blood sample) using INVADER detection reagents (e.g., primary probe, INVADER probe, and FRET cassette). In preferred embodiments, the kit further comprises instructions on how to perform the INVADER assay and specifically how to apply the INVADER detection assay to pooled samples from many individuals, or to “pooled” samples from many cells (e.g., from a biopsy sample) from a single subject.

The present invention further provides assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g., through probe-probe displacement or through an equilibrium between probe/target association and disassociation, or through a combination comprising these mechanisms, [The kinetics of oligonucleotide replacement. Luis P. Reynaldo, Alexander V. Vologodskii, Bruce P. Neri and Victor I. Lyamichev. J. Mol. Biol. 97: 511-520 (2000)], multiple probes can hybridize to the same target, allowing multiple cleavages, and the generation of multiple cleavage products.

In preferred embodiments, control and test samples to be compared in any given experiment are purified by the same method. In particularly preferred embodiments, DNA is quantified following purification, e.g. by PICOGREEN (Molecular Probes, Eugene, Oreg.) assay or A₂₆₀, and before analysis and comparable amounts of the appropriate controls and test samples are added to the respective assays. In particularly preferred embodiments, the amount of DNA added to a test sample is between 10-160 ng/20 μl assay or 3-30 ng/10 assay.

The INVADER assay is suitable for use with a variety of sample types. For example, in some embodiments of the present invention utilizing INVADER assay detection of gene dosage sample types include, but are not limited to, amniocyte cells, cystic hygroma fluid, amniocyte cell culture, amniotic fluid, chorionic villi, fetal urine, fetal skin, and fetal blood (See e.g., Donnenfeld and Lamb, Clin. Lab. Med. 23:457 [2003]; herein incorporated by reference).

In some particularly preferred embodiments, the selection of specific sequences for detection by, and the design of oligonucleotides for use in an INVADER assay is carried out using computational methods. In some embodiments, such methods for the design of oligonucleotides that successfully hybridize to appropriate regions of target nucleic acids under the desired reaction conditions (e.g., temperature, buffer conditions, etc.) for the detection assay. In some embodiments of the present invention, assay design is carried out using INVADERCREATOR software (Third Wave Technologies, Madison, Wis.), which calculates ideal oligonucleotide sequences and reaction conditions for conducting invasive cleavage reactions, e.g., as described in U.S. patent application Ser. No. 09/864,636 and 10/336,446, each incorporated herein in its entirety for all purposes.

In some embodiments, a multistep computational approach is applied to identify sequences that are unique in the human genome. In some preferred embodiments, some of the steps of a computational approach involve the creation of integrated databases of human genomic sequences or sequence variations. Variation in the human genome sequence accounts for a large fraction of observed differences between individuals, and it has been established that common genetic variants underlie susceptibility to many diseases as well as response to therapeutic treatments. To date over 10 million variations have been submitted to over 8 databases in the public domain.

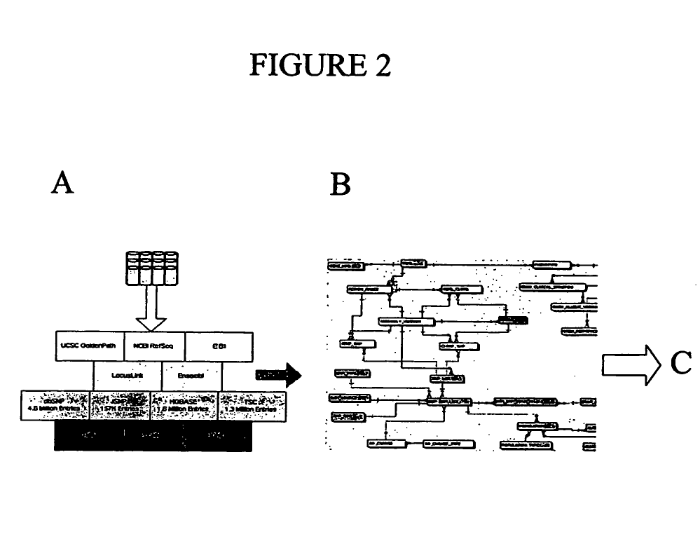

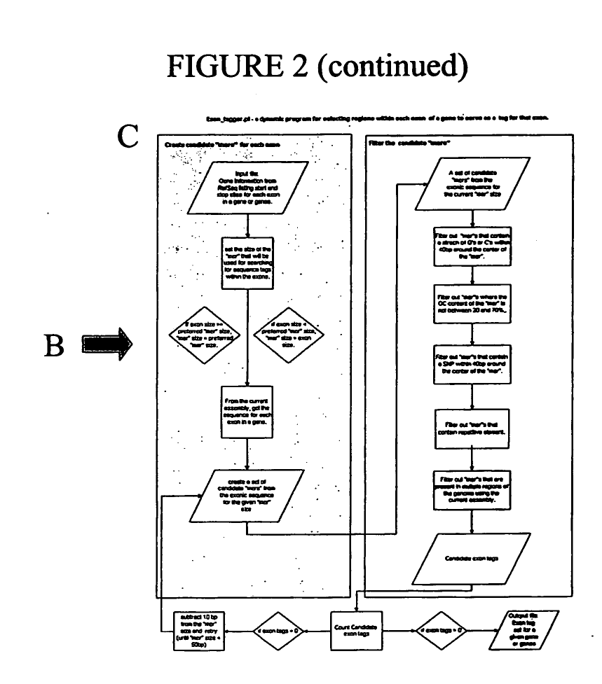

While the number and the mapping of genes and sequence variations differs between public databases, integrating these data points and applying computational algorithms creates a platform to enable the detection of sequences without known variation which mark gene regions. Gene regions may encompass both known or predicted regulatory sites, exons, splice sites and other functional regions. In order to identify these important regions, a multi-step, iterative computational approach can be applied. For example, a program termed EXON TAGGER was designed to identify marker sequences for regions within an exon, which can be used to identify either the complete exon or a part of it. One embodiment of this approach is schematized in FIG. 2.

In a preferred embodiment, the EXON TAGGER computational schema begins with a curated collection of known genes and sequence variations that are integrated into an internal database and mapped to the current assembly of the human genome. The database integrates data sets from diverse sources by applying data normalization techniques, establishing a common vocabulary for labeling data elements, and resolving data inconsistencies through manual curation. The database provides the platform for analyzing gene regions and sequence variants in gene regions. In one particularly preferred embodiment, NCBI's collection of Reference Sequences (RefSeq) serves as the primary source of genes. Secondary sources may include proprietary sequences, computational prediction of coding regions using gene prediction programs, and published literature. Several sources of genetic variation are integrated and include NCBI's database of SNPs (dbSNP), the database of Japanese single nucleotide polymorphisms (JSNP), Human Genome Variation Database (HGVbase), and variants identified in published literature and public databases with a focus on pharmacogenomic variants. The primary source for genome assembly and the accompanying annotation are from the University of California Santa Cruz GoldenPath (UCSC GoldenPath).

In some preferred embodiments, the EXON TAGGER computational schema is designed to accommodate several empirically determined and experimentally significant variables. These factors include, sequence variation within the exon tag, uniqueness of the tag across the genome, and the suitability of any given region for analysis by a nucleic acid-based assay, e.g. the INVADER assay, AS-PCR, or TaqMan. The computational algorithm relies upon an iterative process to identify a marker sequence that will uniquely identify an exon by utilizing a dynamic programming approach. The algorithm also filters marker sequences to identify the “best” candidates for development of a molecular assay by considering the presence of sequence variants and several local sequence content elements. The local sequence content examined includes the presence of repetitive elements and simple repeats, and GC content. Additional analysis using methods known in the art can be applied to identify the presence of pseudogenes, gene duplications, and other homologies that may interfere with the interpretation of the molecular assay results.

Selecting Sequences to Represent Genes:

In some embodiments, it is preferred to obtain sequences with the most annotation. In other embodiments, RefSeq sequences are used to identify genes. In still further embodiments, it is desired to identify exonic regions. RefSeq sequences are curated mRNA sequences which identify the exon start and stop sites of various genes according to the current assembly, in addition to providing sequence annotation identifying the untranslated region (UTR) sites, coding sequence (CDS) start sites and any other annotation associated with the mRNA sequence. At times, more than one reference sequence may exist for a given gene. In this case, there are multiple splice forms of the gene that are known to exist. In some embodiments, to get the most complete form of the gene, the RefSeq sequence that represents the longest sequence is identified by summing the exon start and stop sites listed for the RefSeq sequence. In some embodiments, if there are multiple RefSeq Sequences, the RefSeq sequence with the lowest RefSeq ID is chosen to represent the gene. In other embodiments, all RefSeq splice variants are entered into the exon tagger program and duplicate exon tags are filtered at the end. Duplicate tags between RefSeq entries are removed by removing tags that have 100% identity to another tag (e.g., via pairwise comparison). In the case of duplicate tags, the tag with the lowest RefSeq id is retained. By finding the unique tags, sequences that may be in one splice variant but not another, are not lost by filtering arbitrarily by sequence size. This anchors the rule set back to the actual sequence). This RefSeq sequence is the foundation for the analysis and provides an annotation to find the sequence for each exon in the gene. Candidate genes are identified by their approved gene symbol, LocusLink identifier, reference mRNA accession (NM_#), or reference protein accession (NP_#). Selecting the size of the exon tag:

The size of exons within a gene is highly variable and may be many nucleotides in length or just a few. In a preferred embodiment, exon tags are 91 base pairs in length. If the exon is shorter than 91 base pairs in length the program resets the exon tag size to be the same as the size of the exon. The desired size of the exon tag can be set as a parameter in the EXON TAGGER program to be any given length. Once the exon tag size is determined, a set of tags for the targeted exon is created. These tags are then checked to see if they occur uniquely in the genome and do not appear to have any sequence features that may make them problematic assays (see sections below). If no appropriate exon tags are found, the program resets the exon tag size to the current exon tag size minus 10 and re-tests the new tags. This process continues until the exon tag size is less than 50 bp. If no appropriate exon tags are found for the exon or the exon size is less than 50 base pairs, potential exon tags are examined by hand.

Creating Exon Tags:

Exon tags are created by getting the sequence for the exon from our integrated database. Once the complete sequence for the exon is acquired, the program steps down the exonic sequence to create exon tags using the selected exon tag size. Once an exon tag is created the program steps down the exonic sequence by a specified number of bases (default=5 bp) to create the next exon tag.

Finding a Unique Tag Sequence for an Exon:

In some cases, RefSeq sequences may map to multiple regions within the genome, e.g. when pseudogenes are present, when a given sequence is found to have multiple mappings in genome assembly or partial homology to other regions or members of the same gene family, or may be otherwise incompletely assembled. If significant homology to multiple genomic regions is detected, (e.g., if a gene is duplicated in the genome), the exon tags for the gene may require PCR to amplify the region of interest before testing for each exon tag. Alternatively, in some embodiments, it may be desirable to target such duplicated sequences in order to increase signal generated from a discrete assay. In some embodiments, the EXON TAGGER algorithm can be directed to eliminate candidate exon tags that may contain repetitive element and therefore may be duplicated in the genome. In other embodiments, such repetitive elements may be retained. In some embodiments, candidate exon tags are compared against the current assembly of the genome to verify that each of the candidate tags appears only the anticipated number of times in the genome.

Checking Each Candidate Exon Tag for Assay Ability:

In order to provide the best possible exon tags for each exon, the EXON TAGGER program looks for strings of G's or C's and SNPs within the tag sequence. If the exon tag sequence is found to have SNPs or strings of 5 or more G's or C's within the 40 bp around the center of the exon tag, the candidate exon tag sequence is removed. In addition, the program can be designed to measure GC content within the tag sequence. If desired, the number of G's or C's, as well as the overall GC content, can be modulated. In preferred embodiments, sequences that have more than 70% GC content or less than 20% GC content are removed. In particularly preferred embodiments, sequences having more than 60% GC content or less than 40% GC content are removed.

### EXPERIMENTAL EXAMPLES

The following examples are provided to illustrate certain embodiments of the invention without being intended to limit the invention to the specific applications described.

In the disclosure that follows, the following abbreviations apply: Ex. (Example); Fig. (Figure); ° C. (degrees Centigrade); g (gravitational field); hr (hour); min (minute); olio (oligonucleotide); r×n (reaction); vol (volume); w/v (weight to volume); v/v (volume to volume); BSA (bovine serum albumin); CTAB (cetyltrimethylammonium bromide); HPLC (high pressure liquid chromatography); DNA (deoxyribonucleic acid); p (plasmid); ml (microliters); ml (milliliters); mg (micrograms); mg (milligrams); M (molar); mM (milliMolar); mM (microMolar); pmoles (picomoles); amoles (attomoles); zmoles (zeptomoles); nm (nanometers); kdal (kilodaltons); OD (optical density); EDTA (ethylene diamine tetra-acetic acid); FITC (fluorescein isothiocyanate); SDS (sodium dodecyl sulfate); NaPO4 (sodium phosphate); NP-40 (Nonidet P-40); Tris(tris(hydroxymethyl)-aminomethane); PMSF (phenylmethylsulfonylfluoride); TBE (Tris-Borate-EDTA, ie., Tris buffer titrated with boric acid rather than HCl and containing EDTA); PBS (phosphate buffered saline); PPBS (phosphate buffered saline containing 1 mM PMSF); PAGE (polyacrylamide gel electrophoresis); Tween (polyoxyethylene-sorbitan); Red (REDMOND RED Dye, Epoch Biosciences, Bothell Wash.) Z28 (ECLIPSE Quencher, Epoch Biosciences, Bothell, Wash.); ATCC (American Type Culture Collection, Rockville, Md.); Coriell (Coriell Cell Repositories, Camden, N.J.); DSMZ (Deutsche Sammlung von Mikroorganismen und Zellculturen, Braunschweig, Germany); Ambion (Ambion, Inc., Austin, Tex.); Boehringer (Boehringer Mannheim Biochemical, Indianapolis, Ind.); MJ Research (MJ Research, Watertown, Mass.; Sigma (Sigma Chemical Company, St. Louis, Mo.); Dynal (Dynal A.S., Oslo, Norway); Gull (Gull Laboratories, Salt Lake City, Utah); Epicentre (Epicentre Technologies, Madison, Wis.); Lampire (Biological Labs., Inc., Coopersberg, Pa.); MJ Research (MJ Research, Watertown, Mass.); National Biosciences (National Biosciences, Plymouth, Minn.); NEB (New England Biolabs, Beverly, Mass.); Novagen (Novagen, Inc., Madison, Wis.); Perkin Elmer (Perkin-Elmer/ABI, Norwalk, Conn.); Promega (Promega, Corp., Madison, Wis.); Stratagene (Stratagene Cloning Systems, La Jolla, Calif.); Clonetech (Clonetech, Palo Alto, Calif.) Pharmacia (Pharmacia, Piscataway, N.J.); Milton Roy (Milton Roy, Rochester, N.Y.); Amersham (Amersham International, Chicago, Ill.); and USB (U.S. Biochemical, Cleveland, Ohio). Glen Research (Glen Research, Sterling, Va.); Coriell (Coriell Cell Repositories, Camden, N.J.); Gentra (Gentra, Minneapolis, Minn.); Third Wave Technologies (Third Wave Technologies, Madison, Wis.); PerSeptive Biosystems (PerSeptive Biosystems, Framington, Mass.); Microsoft (Microsoft, Redmond, Wash.); Qiagen (Qiagen, Valencia, Calif.); Molecular Probes (Molecular Probes, Eugene, Oreg.); VWR (VWR Scientific,); Advanced Biotechnologies (Advanced Biotechnologies, INC., Columbia, Md.).

### Example 1

### Measurement of Gene Dosage Using the INVADER Assay

A. DNA Samples

Aneuploidy cell lines were obtained from the Coriell Institute for Medical Research. DNA was isolated using the Gentra Systems, Inc. (Minneapolis, Minn.) PUREGENE DNA Purification Kit (for manual purification) or AUTOPURE LS (for automated purification). The following cell lines were obtained:

TABLE 1
Cell lines and their genotypes.

| Description | Karyotype | Repository # | Cell Type |
|---|---|---|---|
| Trisomy 18 | 48, XXX, +18 | GM03623 | Fibroblast |
| Trisomy 18 | 47, XX, +18 | GM02422 | Amniotic |
| | | | fluid |
| Trisomy 18 | 47, XX, +18 | AG12614 | Fibroblast |
| Trisomy 13 | 47, XX, +13 | AG12070 | Aminotic |
| | | | fluid |
| Trisomy 13 | 47, XY, +13 | GM03330 | Fibroblast |
| Trisomy 13 | 47, XY, +13 | GM02948A | Fibroblast |
| Trisomy 21 | 47, XY, +21 | AG09394 | Lymphoblast |
| Trisomy 21 | 47, XX, +21 | AG13429 | Lymphoblast |
| Trisomy 21 | 47, XX, +21 | AG10098 | Lymphoblast |
| Monosomy 21 | peripheral blood | NA01201(DNA) | Lymphoblast |
| | lymphocyte sample: | | (provided as |
| | 46, XX, −21, | | DNA) |
| | +t(21; 21), | | |
| | lymphoblast culture | | |
| | was 45, XX, −21 | | |
| XYY Syndrome | 47, XYY | GM01250A | Fibroblast |
| XYY Syndrome | 47, XYY | GM09326 | Fibroblast |
| XXXXX | 48, XXXX; 49, | GM05009C | Fibroblast |
| Syndrome | XXXXX | | |
| Iso X Syndrome | 45, X/46, X, i (X) | GM03543 | Lymphocyte |
| | (qter > cen > qter) | | |
| XO Syndrome/ | 45, X/46, X, i (X) | GM13166 | Lymphocyte |
| Turner Syndrome | (qter > cen > qter) | | |
| Turner Syndrome | 45, X | AG08006 | Lymphocyte |
| Aneuploid | 48, XXXX | GM01416E | Lymphocyte |

Control disomic samples were prepared using either the Gentra Systems, Inc. (Minneapolis, Minn.) PUREGENE DNA Purification Kit (for manual purification) or AUTOPURE LS (for automated purification) to purify genomic DNA from whole blood or tissue culture cells. Both kits were used according to the manufacturer's protocols. DNA was prepared from cultured amniocytes using a homebrew phenol chloroform DNA extraction method. Unless stated otherwise, control and test samples to be compared in any given experiment were purified by the same method. DNA was quantified following purification, e.g. by PICOGREEN (Molecular Probes, Eugene, Oreg.) assay or A₂₆₀, before analysis and comparable amounts of the appropriate controls and test samples, ranging from 5-160 ng of DNA, were added to the respective assays.

B. INVADER Oligonucleotide Designs

The EXON TAGGER method (see Description) was used to identify candidate regions as suitable targets for the INVADER assay. The INVADER CREATOR program was then used to design INVADER and probe oligonucleotide sequences. Oligonucleotides comprising appropriate designs were synthesized using conventional procedures. Example 2 contains a description of how the use of this program was modified to result in the selection of candidate target regions with various degrees of chromosomal specificity.

C. INVADER Assay Reagents and Methods

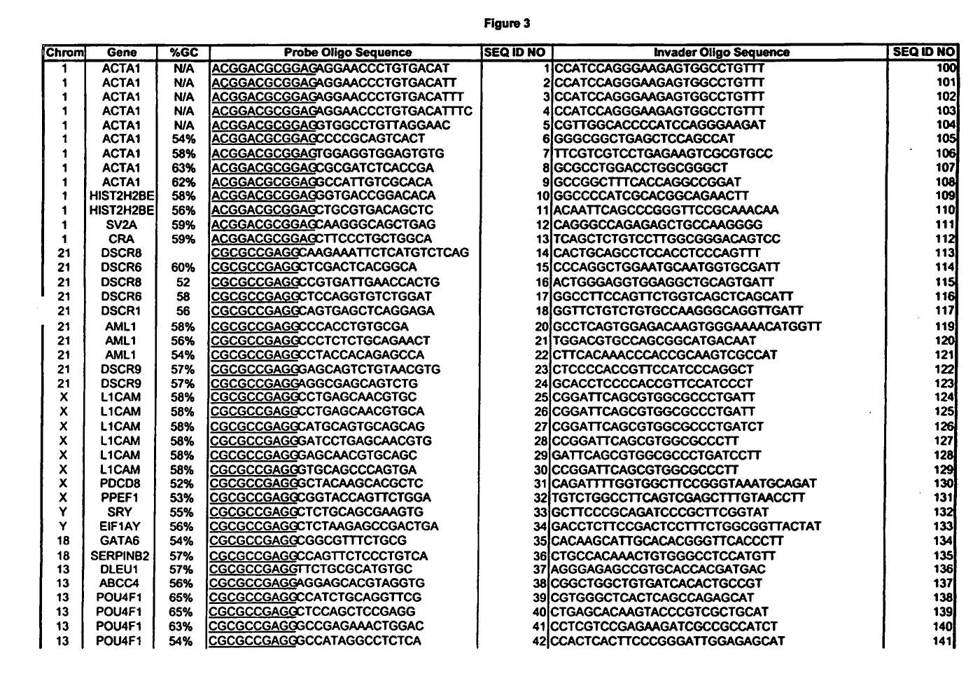

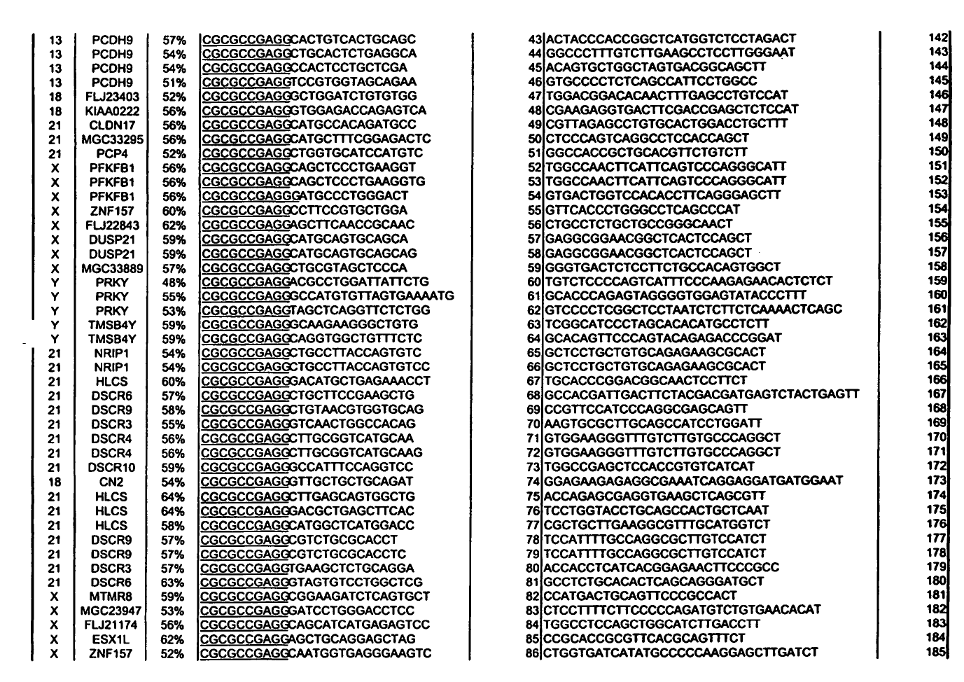

FIG. 3 lists the genes on each chromosome targeted for analysis and the oligonucleotide sequences of the INVADER and probe oligonucleotides used to detect the various genes. For each of these sequences, the 5′ portion (“flap”) is highlighted with underlining. The remaining non-underlined part of the sequences is the 3′ portion (Target Specific Region). Also, fragments that would be generated during an invasive cleavage reaction with these sequences (and the indicated INVADER oligonucleotides) are the underlined sequence (5′ portion) plus the first base from the 3′ portion. These fragments are designed to participate in a second invasive cleavage reaction with a FRET cassette by serving as the INVADER (upstream) oligonucleotide in this second invasive cleavage reaction. All of these probe oligonucleotides contain hexanediol as a 3′ blocking group.

INVADER assays were set up to determine chromosome copy number as follows. Target DNA was provided as genomic DNA prepared as described above. Biplex INVADER reactions (e.g. as shown in FIG. 1), in which a chromosome-specific oligonucleotide set was biplexed with oligonucleotides directed to an internal control (e.g. a portion of the α-actin gene (ACTA1), with oligonucleotide sequences of SEQ ID NOs:1 and 100; ACTA1; FIG. 3), were carried out in a final volume of 20 μl in a 96-well microplate. The chromsomes and genes targeted, their chromsomal location (“cytoband”), and the Genbank accession number consulted for assay design are listed below.

TABLE 2
Chromosomes and genes targeted in the INVADER assay.

| Chromosome | Gene | Genbank Accession # | Cytoband |
|---|---|---|---|
| 21 | STCH | NM_006948 | 21q11 |
| | DSCR6 | NM_018962 | 21q22.13 |
| | AML1 exon 1 | NM_001754 | 21q22.12 |
| | AML1 exon 4 | NM_001754 | 21q22.12 |
| X | AR | NM_000044 | Xq12 |
| | L1CAM | NM_000425 | Xq28 |
| | PDCD8 | NM_004208 | Xq26.1 |
| | PPEF1 | NM_006240 | Xp22.13 |
| Y | SRY | NM_003140 | Yp11.31 |
| | EIflAY | NM_004681 | Yq11.222 |
| 18 | GATA6 | NM_005257 | 18q11.2 |
| | SERPIN B | NM_002575 | 18q22.1 |
| 13 | CCNA | NM_003914 | 13q13.3 |
| | ING1 | NM_005537 | 13q34 |
| | DLEU1 | NM_005887 | 13q14.2 |

Aliquots of 10 μl of each sample (genomic DNA, final amounts ranging between 5 ng to 160 ng per reaction) or no target control (100 ng/μl tRNA) were added to the appropriate wells and then overlaid with 25 μl mineral oil. Samples were denatured at 95° C. for 5 minutes and then cooled to 75° C. A 10 μl aliquot of the following INVADER reaction mix was then added to each well and mixed by pipetting:

TABLE 3

INVADER assay reaction components

| | |
|---|---|
| Amount | |
| per | Final |

| | | |
|---|---|---|
| Component | reaction | concentrations |

| | | | |
|---|---|---|---|
| DNA reaction buffer 1 (14% | 5 | μl | 3.5% PEG, |
| PEG, 40 mM MOPS, pH 7.5, | | | 10 mM MOPS, |
| 56 mM MgCl₂, 0.02% ProClin | | | 14 mM MgCl₂ |
| 300) | | | |
| Chromosome Specific | 1 | μl | 10 pmol |
| Primary probe/INVADER | | | probe, 1 pmol |
| oligo (10 μM/1 μM) | | | invader |
| Internal Control Primary | 1 | μl | 10 pmol probe, |
| Probe/Invader (10 μM/1 μM) | | | 1 pmole |
| | | | invader |
| FAM FRET (10 μM) SEQ ID | 0.5 | μl | 5 pmol |
| NO: 199 [Fam-TCT-Z28-AGCCG | | | |
| GTTTTCCGGCTGAGACCTCGGCGCG- | | | |
| hex] | | | |
| RED FRET (10 μM) SEQ ID | 0.5 | μl | 5 pmol |
| NO: 200 [Red-TCT-Z28-TCGGC | | | |
| CTTTTGGCCGAGAGACTCCGCGTCCG | | | |
| T-hex] | | | |
| CLEAVASE X enzyme (40 ng/ | 1 | μl | 40 ng |
| μl) in CLEAVASE dilution | | | |
| buffer | | | |
| RNAse free water | 12 1 | μl | |

Reactions were incubated at 63° C. for 4 hours and then cooled to 4° C. prior to scanning in a CYTOFLUOR 4000 fluorescence plate reader (Applied Biosystems, Foster City, Calif.). The settings used were: 485/20 nm excitation/bandwidth and 530/25 nm emission/bandwidth for FAM dye detection, and 560/20 nm excitation/bandwidth and 620/40 nm emission/bandwidth for RED dye detection. The instrument gain was set for each dye so that the No Target Blank produced between 100-250 Relative Fluorescence Units (RFUs). NOTE: Because the optimal gain setting can vary between instruments, adjust the gain as needed to give the best signal/background ratio (sample raw signal divided by the No Target Control signal) or No Target Control sample readings of ˜100 RFUs. Fluorescence microplate readers that use a xenon lamp source generally produce higher RFUs. For directly reading the microplates, the probe height of, and how the plate is positioned in, the fluorescence microplate reader is adjusted according to the manufacturer's recommendations.

The raw data that is generated by the device/instrument is used to measure the assay performance (real-time or endpoint mode). The equations below provide how FOZ (Fold Over Zero), and other values are calculated. NTC in the equations below represents the signal from the No Target Control.

FOZ or Signal/No Target
FOZ_{Dye1}=(RawSignal_{Dye1}/NTC_{Dye1})
FOZ_{Dye2}=(RawSignal_{Dye2}/NTC_{Dye2})
In the following examples, FOZ_{Dye1} corresponds to the signal from the chromosome-specific assay, and FOZ_{Dye2}, to that from the internal control assay. The two FOZ values (i.e. chromosome-specific and internal control) for each sample were used to calculate the chromosome-specific: internal control Ratio as follows:

$$Ratio = \frac{( Net chromosome specific FOZ )}{( Net internal control FOZ )}$$

where Net FOZ=FOZ−1
To determine the ratio normalized to two copies per genome, the following calculation is performed with the Net FOZ ratios.
Normalized Ratio=(Ratio/Average 2−copy ratio)×2
C. Limit of Detection (LOD) of INVADER Assays

Experiments were conducted to examine the effect of varying DNA concentration on assay performance and the ability to discriminate genotypes. In particular, the effects of limiting (i.e., 5 ng) genomic DNA were addressed.

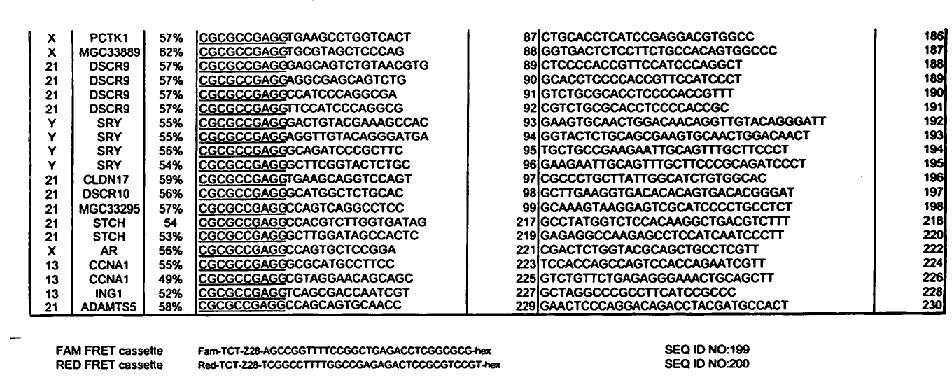

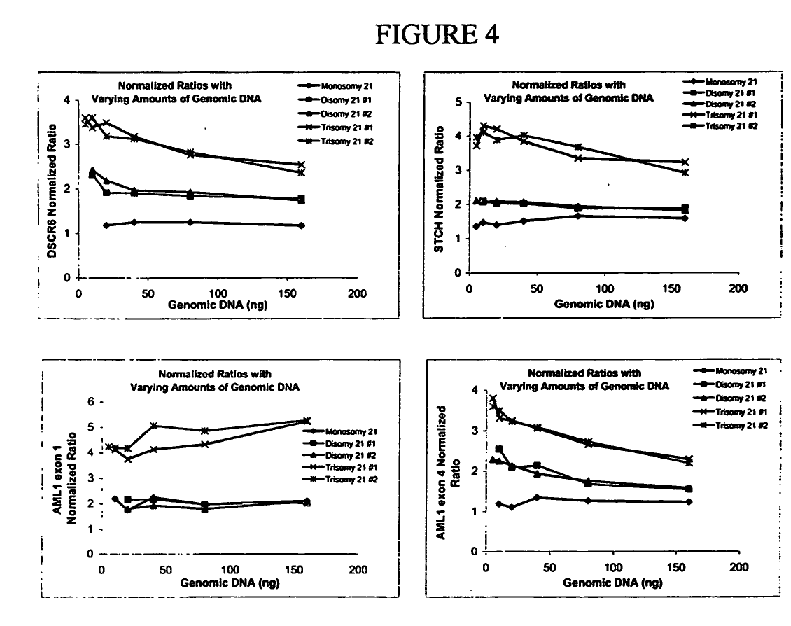

INVADER assays were set up as described in Example 1B, with the final amounts of genomic DNA ranging from 0-160 ng. DNA was quantified using the PICOGREEN test (Molecular Probes, Eugene Oreg.). Assays were designed to genes on chromosome 21: DSCR 6 (SEQ ID NOs: 17 and 116); STCH (SEQ ID NO2: 217 and 218) and AML exons 1 (SEQ ID NOs: 20 and 119) and 4 (SEQ ID NOs: 22 and 121). The results are presented in FIG. 4 and indicate that the assays were informative across the range of DNA concentrations tested at or above 10 ng. Samples containing just 5 ng of genomic DNA failed to yield FOZ values above 1.15, which were therefore excluded from consideration as being below the limit of robust detection. DNA concentrations greater than 160 ng were not tested in this experiment.

D. Results of INVADER Assays to Detect Variations in Gene Dosage

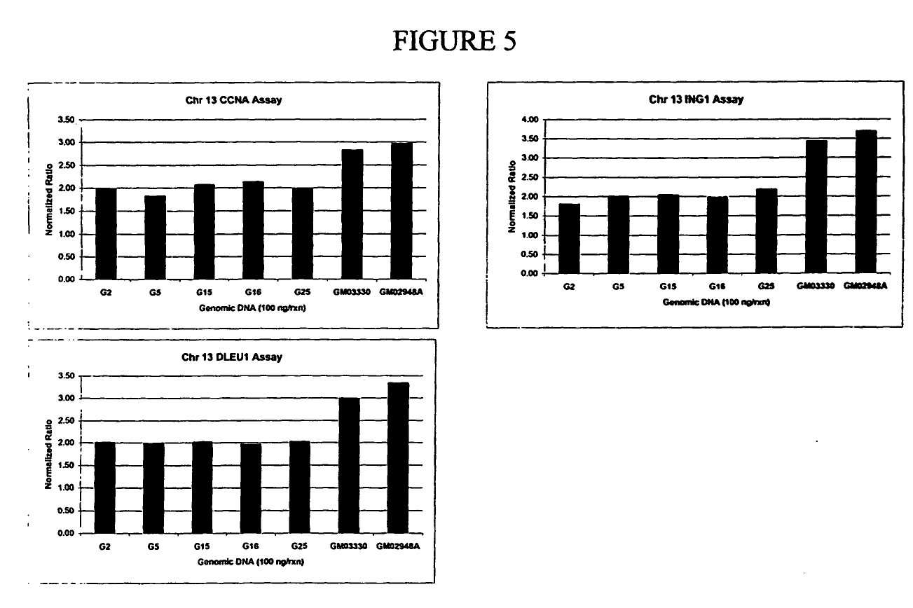

The results of these experiments from reactions directed to chromosomes 13, 18, 21, X and Y are presented in FIGS. 5-9, respectively. The samples are aligned along the X-axis and grouped according to sample type (i.e. disomy or trisomy). The normalized ratio is indicated along the Y-axis as denoted in the legend.

i. Analysis of Chromosome 13 Copy Number

FIG. 5 presents the results of the analysis of INVADER assays to determine copy number of loci carried on chromosome 13: deleted in lymphocytic leukemia, 1, (DLEU; SEQ ID NOs: 37 and 136); cyclin A1 (CCNA; SEQ ID NOs: 223 and 224); and inhibitor of growth family, member 1 (ING1; SEQ ID NOs: 227 and 228). The samples tested are listed along the X-axis and labeled. Samples G2, 5, 15, 16, and 25 were obtained from normal individuals presumed to be disomic for chromosome 13; samples GM03330 and GM02948A were obtained from Coriell and are trisomic for chromosome 13 (see Table 1).

These results demonstrate that the INVADER assay can readily discriminate disomy (normal, 2 copy) and trisomy (abnormal, 3 copy) cases at these chromosome 13 loci.

ii. Analysis of Chromosome 18 Copy Number

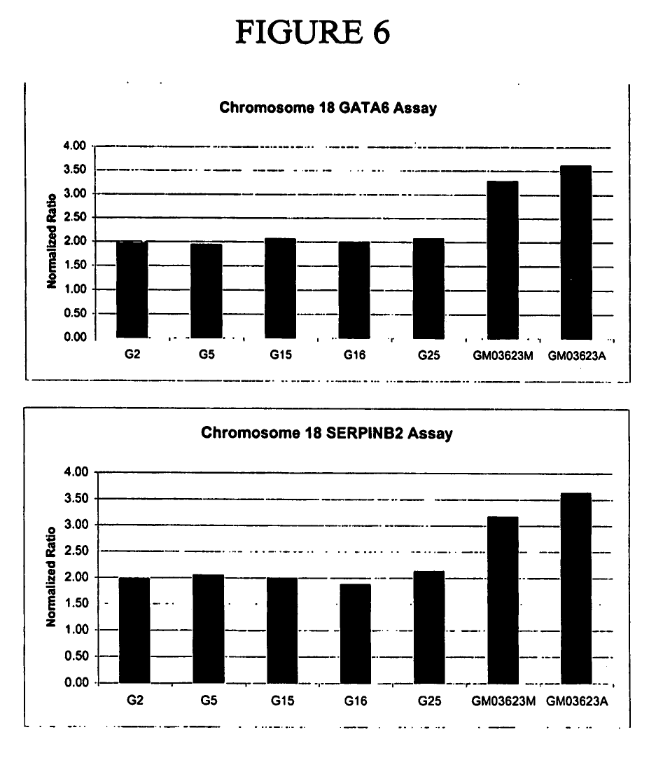

FIG. 6 presents the results of the analysis of INVADER assays to determine copy number of loci carried on chromosome 18: GATA-binding protein 6; (GATA6, SEQ ID NOs: 35 and 134) and serine (or cysteine) proteinase inhibitor, clade B (ovalbumin), member 2 (SERPINB2; SEQ ID NOs: 36 and 135). The samples tested are listed along the X-axis and labeled. Samples G2, 5, 15, 16, and 25 were obtained from normal individuals presumed to be disomic for chromosome 18; sample GM03623 was obtained from Coriell and is trisomic for chromosome 18 (see Table 1); M and A refer to “manual” and “autopure” and describe the procedures used to isolate the DNA from the Coriell cell line.

These results demonstrate that the INVADER assay can readily discriminate disomy (normal, 2 copy) and trisomy (abnormal, 3 copy) cases at these chromosome 18 loci.

iii. Analysis of Chromosome 21 Copy Number

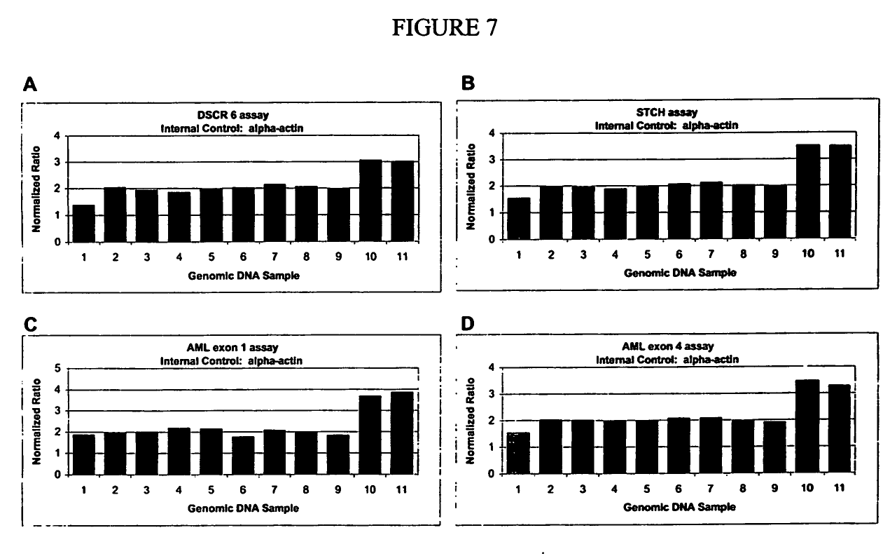

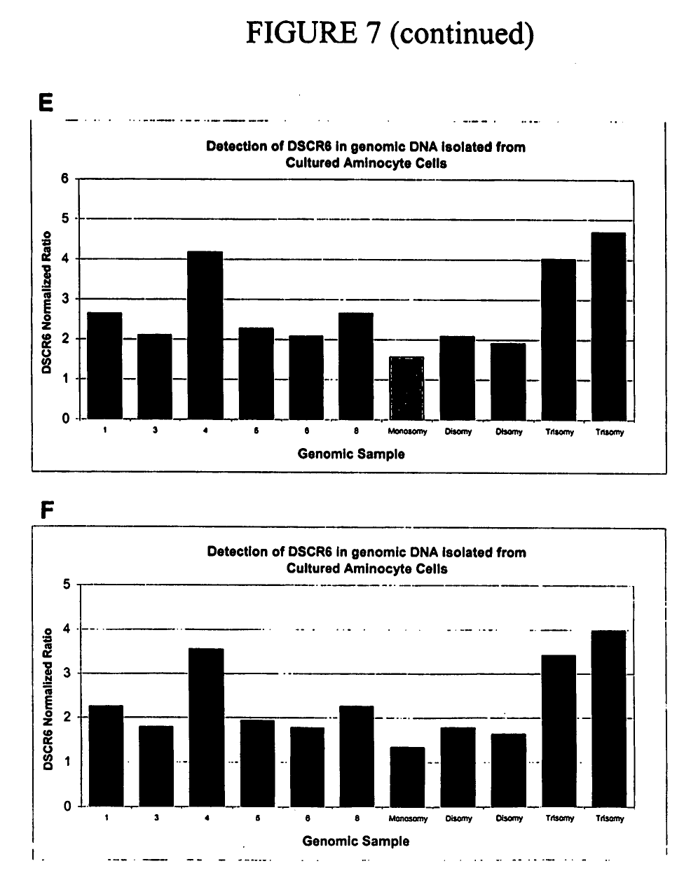

FIGS. 7 A-D present the results of the analysis of INVADER assays carried out on DNA extracted from blood to determine copy number of loci carried on chromosome 21: DSCR6 (Downs' syndrome critical region 6; SEQ ID NOs: 17 and 116), AML exon 1 (runt-related transcription factor 1 (acute myeloid leukemia 1, aml1 oncogene)), SEQ ID NOs: 20 and 119), STCH (stress 70 protein chaperone; SEQ ID NOs: 217 and 218); and AML exon 4 ((runt-related transcription factor 1 (acute myeloid leukemia 1, aml1 oncogene)), SEQ ID NOs: 22 and 121; see Table 2 for chromosome locations). The samples tested are listed along the X-axis and labeled. Samples 2-9 were obtained from normal individuals presumed to be disomic for chromosome 21, Sample 1 was obtained from Coriell (NA01201) and is monosomic for chromosome 21 and Samples 10 and 11 were obtained from Coriell (AG13429 and AG09394 respectively) and are trisomic for chromosome 21.

FIGS. 7 E-F present the results of the analysis of INVADER assays carried out on blinded genomic DNA samples isolated from cultured amniocytes to determine copy number of loci carried on chromosome 21: DSCR6 (Downs' syndrome critical region 6; SEQ ID NOs: 17 and 116); STCH (stress 70 protein chaperone; SEQ ID NOs: 217 and 218). Both of these INVADER assays reported Samples 1-5 to be disomic for chromosome 21 and Sample 6 to be trisomic for chromosome 21. INVADER assay results corresponded with karyotype results.

These results demonstrate that the INVADER assay can readily discriminate disomic (normal, 2 copy) and monosomic or trisomic mutant karyotypes at these chromosome 21 loci.

iv. Analysis of X and Y Chromosome Copy Number

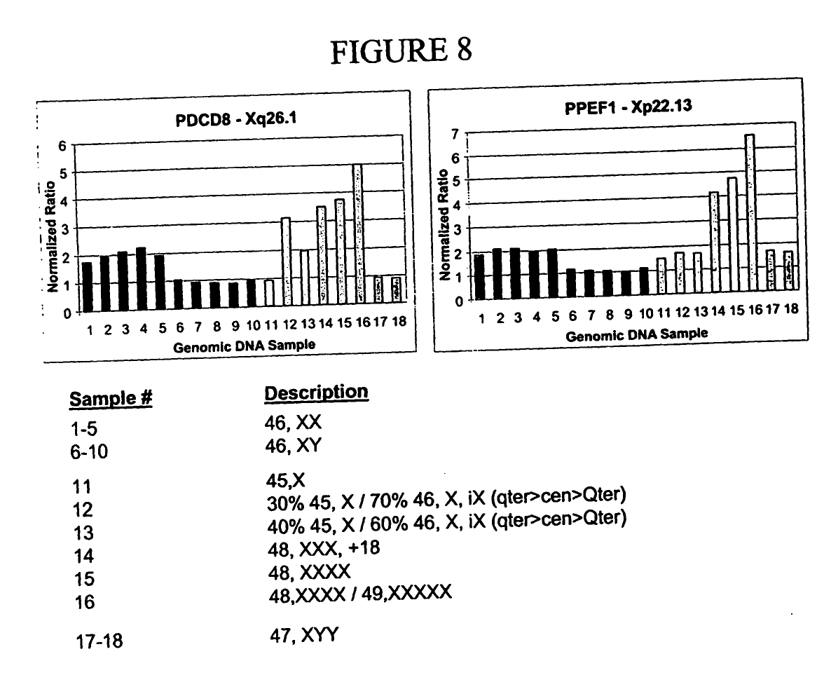

FIG. 8 presents the results of the analysis of INVADER assays to determine copy number of two different loci carried on different arms of the X chromosome: PDCD8 (programmed cell death; Xq26.1; SEQ ID NOs: 31 and 130), and PPEF1 (protein phosphatase, EF-hand calcium-binding domain 1; Xp22.13; SEQ ID NOs: 32 and 131). The samples tested are listed along the X-axis and labeled. In addition to testing 10 genomic DNA samples isolated from the blood of normal individuals (5 each from XX females [samples 1-5] and XY males [samples 6-10]), genomic DNA from individuals with various X/Y anomalies was tested (see Table 1).

These results demonstrate that the INVADER assay can readily discriminate normal XX individuals from normal XY individuals. In addition, the following anomalous genotypes were analyzed: 45X (sample 11; Coriell No. AG08006); 2 45X/46X (iso)X samples, both qter>cen>qter, (i.e. the q arms are duplicated, and the p arm is absent) (sample 12, GM13166: 30% 45, X/70% 46, X(iso)X and sample 13, GM03543: 40% 45, X/60% 46, X(iso)X; 48 XXX, +18 (sample 14; GM03263), 48XXXX (sample 15; GM01416E), 48 XXXX/49XXXXX (sample 16; GM05009C) and two 47 XYY samples (samples 17-18; GM01250A and GM09324). Samples 12 and 13 appear to contain more copies of the q arm of the X chromosome (FIG. 8A) than of the p arm (FIG. 8b), consistent with the presence of a q duplicated isochromosome in those samples. The remaining samples contain additional copies of the X chromosome

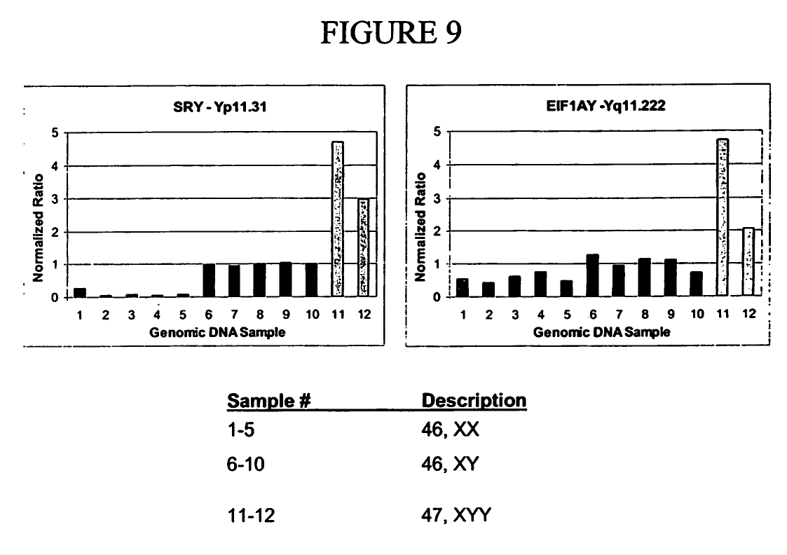

FIG. 9 shows the results of samples analyzed at two different loci on the Y chromosome: SRY (sex determining region Y, SEQ ID NOs: 33 and 132) and EIF1AY (eukaryotic translation minitiation factor 1A, Y chromosome, SEQ ID NOs: 34 and 133). Samples 1-5 are normal XX samples; and 6-10, normal XY samples. Samples 11-12 contain XYY samples, as in samples 17 and 18 of FIG. 8.

The results in FIG. 9 indicate that, as expected, the normal XX samples contain no copies of the Y chromosome. However, the EIF1AY probe set (SEQ ID NOs: 34 and 133) appears to be somewhat less specific for the Y chromosome than does the SRY probe set, suggesting that it may be desirable to examine alternative Y chromosome sequences to ensure specificity. Moreover, the XYY samples appear to contain more than just the two copies of Y anticipated, further suggesting that homologous sequences may be being detected by the probe sets or that there was a potential problem with the preparation of this genomic DNA sample. It is noteworthy, however, that the combination of results presented in FIGS. 8 and 9 lead to a consistent determination of the number of X chromosomes in these two samples and to the definitive presence of aneuploidy vis a vis the Y chromosome.

### Example 2

### Use of The EXON TAGGER Program to Identify Target Regions

A. Target Sequences in the DSCR Gene on Chromosome 21

The EXON TAGGER approach was used to identify appropriate, unique 50-mer sequences in the DSCR 6 (Downs Critical Region) gene, on chromosome 21. An initial analysis was done using the June, 2002 human genome assembly. INVADER assays were designed to this sequence with the probe and INVADER oligonucleotides comprising SEQ ID NOs: 15 and 114, respectively. Assays were carried out as described in Example 1.

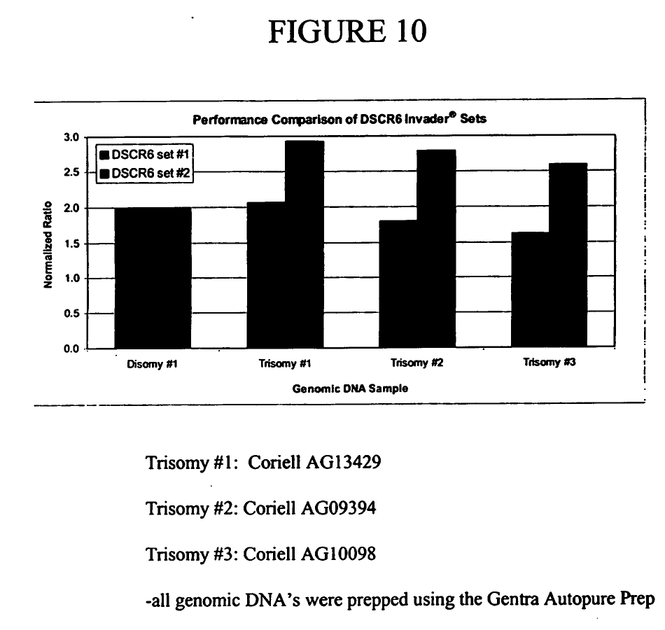

While these oligonucleotide sets appeared to detect the targeted sequence in the DSCR 6 gene, when a sample known to be trisomic for chromosome 21 was tested, the INVADER assay failed to detect a significant increase in signal, as would be expected in the presence of an additional copy of the gene (FIG. 10). It was discovered that the DSCR6 50 mer sequence was part of a SINE (Alu) repeat and was highly homologous to regions on several different chromosomes. The megaBLAST criteria for removing a potential 50mer were set so that only sequences that had 100% homology to other regions were removed, and allowed 50mers with </=99% homology to still remain. The presence of this additional homology had the effect of elevating the total signal such that small differences (e.g. of 50%) were not readily distinguished.

The EXON TAGGER analysis was repeated, using a subsequent genome assembly, the November 2002 human genome assembly, and another probe set was designed to a different 50mer candidate sequence but still within the DSCR6 gene. Assays were carried out as described above and in Example 1.

The results comparing the performance of the initial and redesigned oligonucleotide sets for detecting a region in the DSCR 6 gene are presented in FIG. 10. These results indicate that the INVADER assay designed to target a unique sequence in the DSCR 6 gene can readily discriminate small differences such that samples containing 2 or 3 copies of the DSCR 6 can be distinguished from one another.

B. INVADER Assay Design Screen

Success of INVADER assay designs was based on a clear distinction of disomy and trisomy (or other anomalous karyotypes, as for the X and Y chromosomes) samples, background counts of less than 250, and signal over background values of at least 1.5 using 100 ng of gDNA. Four out of 14 designs (29%) were considered successful, all to chromosome 21 and designed using the initial EXON TAGGER criteria were suitable for distinguishing disomic from trisomic samples. The EXON TAGGER parameters were then changed to include a repetitive element screen and the megaBLAST criteria were set to accept only sequences with <=80% homology to another region in the human genome. These criteria were implemented for the EXON TAGGER data generated for the chromosomes X, Y, 13 and 18. Applying the revised criteria, 14 out of 15 subsequent designs (93%) were deemed to be successful.

### Example 3

### Measurement of Gene Dosage in the Presence of Mock Maternal Genomic DNA Contamination

In certain applications, it may be desirable to measure gene dosage in certain chromosomal samples in the presence of contaminating chromosomal samples from a different source or of a different type, for example fetal or embryonic samples in the presence of contaminating maternal material. To this end, the impact of contaminating disomic DNA, e.g. from a normal individual, on the measurement of chromosome copy number of disomic or trisomic samples was assessed.

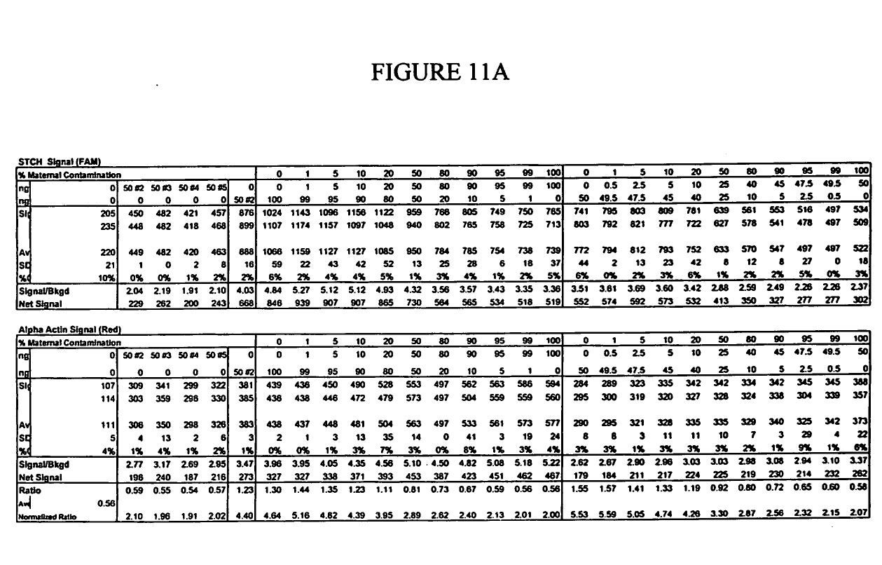

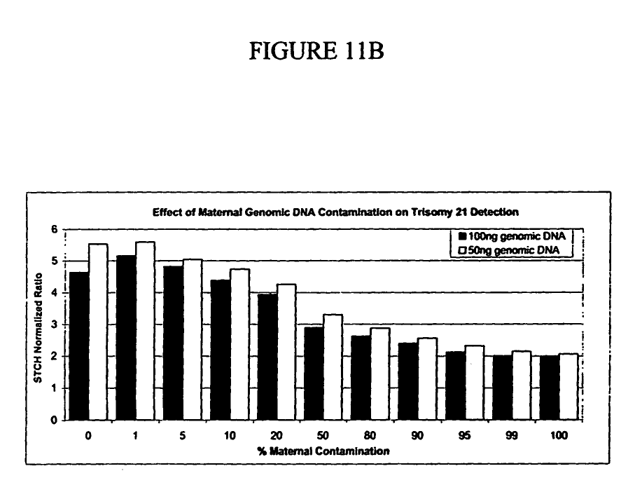

The results are presented in FIG. 11 A-D. Four different loci on chromosome 21 were used as targets to test the effects of combining disomic genomic DNA with trisomic (for chromosome 21) genomic DNA. Reactions were set up and carried out as in Example 1. In each case, the percentage of disomic and trisomic genomic DNA was varied from 0-100%, with the total DNA added to the reaction limited to 50 ng or 100 ng. Normalized ratios elevated significantly above 2 indicate the presence of aneuploid samples, i.e. samples containing an additional copy of the genes examined. These results indicate that the presence of trisomic DNA can be detected in a significant background of disomic or “contaminating” DNA.

### Example 4

### Measurement of Gene Dosage Using Two Invader Assays Per Targeted Chromosome

In certain applications, it may be desirable to measure gene dosage in a sample by using two Invader assays per targeted chromosome and two internal control assays, thus providing double coverage on each of the chromosomes tested.

A. INVADER Assay Reagents and Methods

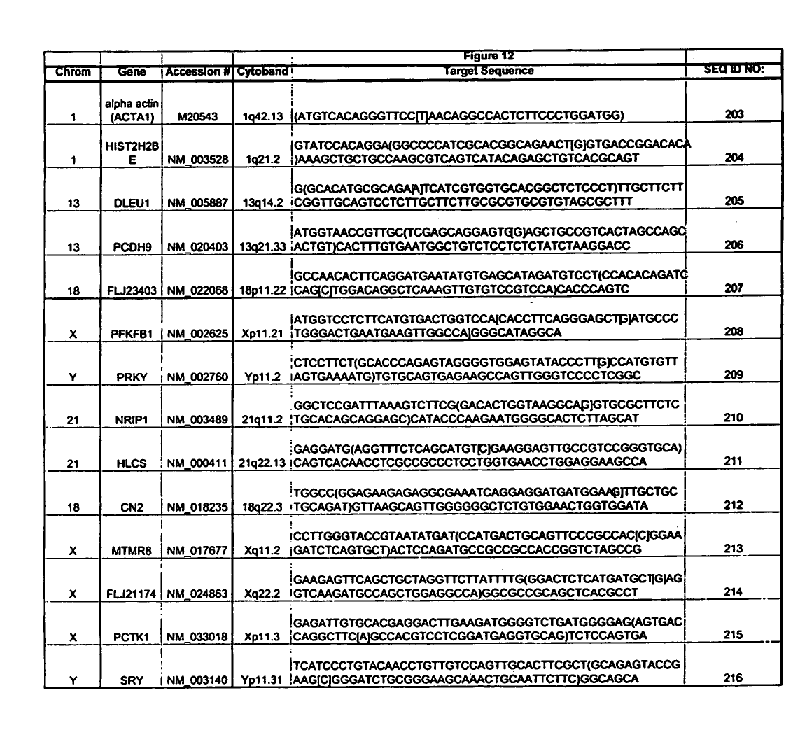

FIGS. 12 and 3 list the genes on each chromosome targeted for analysis and the oligo nucleotide sequences of the INVADER and probe oligonucleotides used to detect the various genes. In this example, Invader probe sets were designed to 2 target regions per chromosome. The target specific probes for chromosomes 13, 18, 21, X and Y contained the same arm (arm 1, CGCGCCGAGG; SEQ ID NO: 201) and utilized the corresponding FAM FRET cassette (SEQ ID NO. 421). The internal control probes for chromosome 1 contained the same arm (arm 3, ACGGACGCGGAG; SEQ ID NO: 202) and utilized the RED FRET cassette (SEQ ID NO. 200). INVADER assays were set up to determine chromosome copy number as follows. Target DNA was provided as genomic DNA prepared as described above. Biplex INVADER reactions (e.g. as shown in FIG. 1), in which the chromosome-specific oligonucleotide sets were used with oligonucleotides directed to a internal controls (e.g. a portion of the α-actin (ACTA1) gene and HIST2HBE), were carried out in a final volume of 10 μl in a 96-well microplate. The chromsomes and genes targeted, their chromsomal location (“cytoband”), and the Genbank accession number consulted for assay design are listed in FIG. 12.

Aliquots of 5 μl of each sample (genomic DNA, final amounts ranging between 3 ng to 30 ng per reaction) or no target control (100 ng/μl tRNA) were added to the appropriate wells and then overlaid with 15 μl mineral oil. Samples were denatured at 95° C. for 5 minutes and then cooled to either 63° C. or 75° C. A 5 μl aliquot of the following INVADER reaction mix was then added to each well and mixed by pipetting:

TABLE 4

INVADER assay reaction components

Final

| | |
|---|---|
| Amount | concentrations |
| per | (in 10 μl |

| | | |
|---|---|---|
| Component | reaction | reaction |

| | | | |
|---|---|---|---|
| DNA reaction buffer 1 (14% | 2.5 | μl | 3.5% PEG, |
| PEG, 40 mM MOPS, pH 7.5, | | | 10 mM MOPS, |
| 56 mM MgCl₂, 0.02% ProClin | | | 14 mM MgCl₂ |
| 300) | | | |
| Chromosome Specific | 1 | μl | 0.5 μM probe, |
| Primary probe/INVADER | | | 0.05 μM |
| oligo/FAM FRET SEQ ID NO: | | | Invader oligo, |
| 199 [Fam-TCT-Z28-AGCCGGTTTT | | | 0.25 μM FRET |
| CCGGCTGAGACCTCGGCGCG-hex] | | | |
| (5 μM/0.5 μM/2.5 μM) | | | |
| Internal Control Primary | 1 | μl | 0.5 μM probe, |
| probe/INVADER oligo/RED | | | 0.05 μM |
| FRET SEQ ID NO: 200 [Red- | | | Invader oligo, |
| TCT-Z28-TCGGCCTTTTGGCCGAGA | | | 0.25 μM FRET |
| GACTCCGCGTCCGT-hex] | | | |
| (5 μM/0.5 μM/2.5 μM) | | | |
| CLEAVASE X enzyme | 0.5 | μl | 2 ng/μl |
| (40 ng/μl) in CLEAVASE | | | |
| dilution buffer | | | |

Reactions were incubated at 63° C. for 4 hours and then cooled to 4° C. prior to scanning in a CYTOFLUOR 4000 fluorescence plate reader (Applied Biosystems, Foster City, Calif.). The settings used were: 485/20 nm excitation/bandwidth and 530/25 nm emission/bandwidth for FAM dye detection, and 560/20 nm excitation/bandwidth and 620/40 nm emission/bandwidth for RED dye detection. The instrument gain was set for each dye so that the No Target Blank produced between 100-250 Relative Fluorescence Units (RFUs). Some of the microplates were also read in the Genios FL Plate reader (Tecan, Research Triangle Park, N.C.). The settings used were: 485/535 nm excitation/emission for FAM dye detection, and 560/612 nm excitation/emission for RED dye detection. The instrument gain was set for each dye so that the No Target Blank produced between 1000-2000 Relative Fluorescence Units (RFUs). NOTE: Because the optimal gain setting can vary between instruments, adjust the gain as needed to give the best signal/background ratio (sample raw signal divided by the No Target Control signal) or No Target Control sample readings. For directly reading the microplates, the probe height of, and how the plate is positioned in, the fluorescence microplate reader may need to be adjusted according to the manufacturer's recommendations.

The raw data that is generated by the device/instrument is used to measure the assay performance (real-time or endpoint mode). The equations below provide how FOZ (Fold Over Zero), and other values are calculated. NTC in the equations below represents the signal from the No Target Control.

FOZ or Signal/No Target
FOZ_{Dye1} (RawSignal_{Dye1}/NTC_{Dye1})
FOZ_{Dye2}=(RawSignal_{Dye2}/NTC_{Dye2})
In the following examples, FOZ_{Dye1} corresponds to the signal from the chromosome-specific assays, and FOZ_{Dye2}, to that from the internal control assays. The two FOZ values (i.e. chromosome-specific and internal control) for each sample were used to calculate the chromosome-specific: internal control Ratio as follows:

$$Ratio = \frac{( Net chromosome specific FOZ )}{( Net internal control FOZ )}$$

where Net FOZ=FOZ−1
To determine the ratio normalized to two copies per genome, the following calculation is performed with the Net FOZ ratios.
Normalized Ratio=(Ratio/Control 2−copy ratio)×2
where the control is a normal female genomic DNA sample or pool of normal female gemomic DNA samples. To determine normalized ratio values to one copy per genome (i.e. Chromosome Y), normal male genomic DNA is used as the control and the ratio is multiplied by 1.
B. Limit of Detection (LOD) of INVADER Assays

Experiments were conducted to examine the effect of varying DNA concentration on assay performance and the ability to discriminate genotypes. In particular, the effects of limiting (i.e., 3 ng) genomic DNA were addressed.

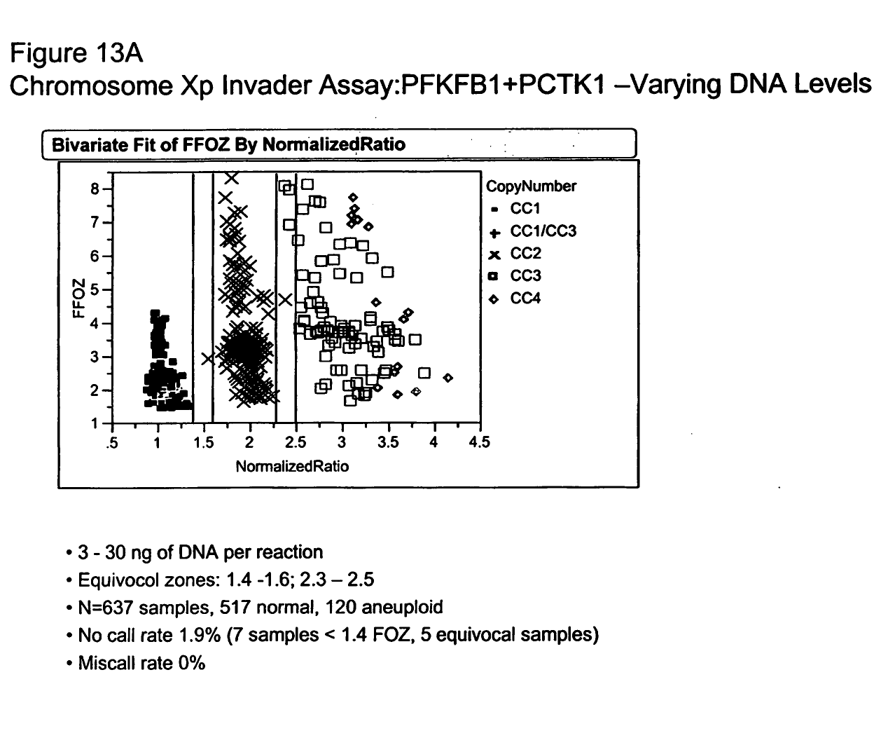

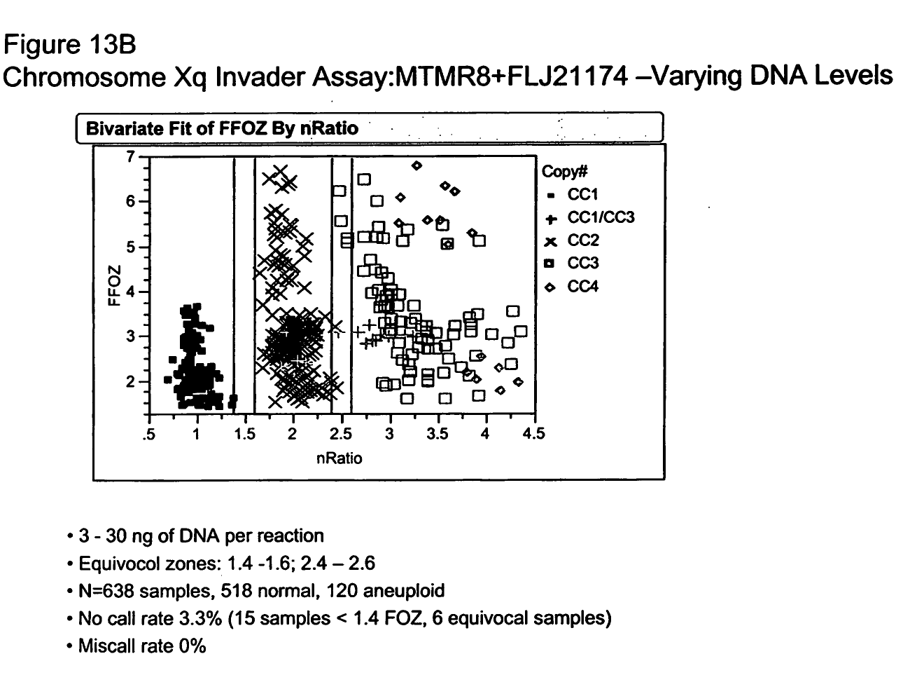

INVADER assays were set up as described in Example 4A using DNA samples described in Example 1A. The CC1/CC3 gDNA was isolated from mosaic samples obtained from Coriell (GM13166 and GM03543) that contained either 30% 45(X), 70% 46 (XiX) or 40% 45 (X), 60% 46 (XiX) respectively. The final amounts of genomic DNA ranged from 3-30 ng. DNA was quantified using the PICOGREEN test (Molecular Probes, Eugene Oreg.). Assays were designed to 4 different genes on chromosome X. Two of the genes are located on the Xp arm PFKFB1 (SEQ ID NOs: 53 and 152); PCTK1 (SEQ ID NOs: 87 and 186), and two of the genes are located on the Xq arm MTMR8 (SEQ ID NOs: 82 and 181); FLJ2174 (SEQ ID NOs: 84 and 183). The internal control genes used in this example were ACTA1 (SEQ ID NOs: 1 and 100); and HIST2HBE (SEQ ID NOs: 10 and 109). The results are presented in FIG. 13 and show the Invader assays can distinguish 1, 2 and 3 or greater copies of X across the range of DNA concentrations tested with the exception of the 3 ng genomic DNA samples. Samples that generated FOZ values less than 1.4 or fell into the equivocal zones were designated as No Calls. Many of the samples containing 3 ng of genomic DNA failed to yield FOZ values above 1.4, which were therefore excluded from consideration as being below the limit of robust detection. DNA concentrations greater than 30 ng were not tested in this experiment. The recommended amount of DNA is 10 ng for the assays that contain 2 assays per targeted chromosome.

The methods described for FIG. 13 were used to evaluate the assay performance for the other targeted chromosomes (13, 18, 21, Y). Samples that did not call correctly (false negatives or false positives) were labeled as Miscalls.

The methods described for FIG. 13 were used to evaluate the assay performance for the other gene targets on chromosomes 13, 18, 21, and Y. The gene targets are listed in FIG. 12. The results of the assay performance are shown in Table 5.

TABLE 5
Summary of Assay performance with varying DNA concentrations

| | | 10 ng DNA/reaction | | | 5-20 ng DNA/reaction | | | 3-30 ng DNA/reaction | | |
|---|---|---|---|---|---|---|---|---|---|---|
| Chrom | Assays | N (total) | No Call | Miscall | N (total) | No Call | Miscall | N (total) | No Call | Miscall |
| 13 | DLEU1 + PCDH9 | 243 | 6 | 0 | 354 | 9 | 0 | 462 | 13 | 2 |
| 18 | FLJ23403 + CN2 | 243 | 1 | 0 | 354 | 1 | 0 | 462 | 3 | 2 |
| 21 | NRIP1 + HLCS | 243 | 6 | 4 | 354 | 8 | 4 | 462 | 12 | 7 |
| Xp | PFKB1 + PCTK1 | 418 | 2 | 0 | 529 | 3 | 0 | 637 | 12 | 0 |
| Xq | MTMR8 + FLJ21174 | 419 | 1 | 0 | 530 | 4 | 0 | 638 | 21 | 0 |
| Y | SRY + PRKY | 420 | 5 | 1 | 531 | 7 | 2 | 639 | 8 | 2 |
| Total | | 1986 | 21 | 5 | 2652 | 32 | 6 | 3300 | 69 | 13 |

Many of the samples containing 3 ng of genomic DNA failed to yield FOZ values above 1.4, which were therefore excluded from consideration as being below the limit of robust detection. DNA concentrations greater than 30 ng were not tested in this experiment. The recommended amount of DNA is 10 ng for the assays that contain 2 assays per targeted chromosome.

C. Assay Performance with Sample Mixtures

Experiments were conducted to examine the effect of sample mixtures on assay performance and the ability to measure gene dosage (i.e. fetal or embryonic samples in the presence of contaminating maternal material). To this end, the impact of contaminating disomic DNA, e.g. from a normal individual, on the measurement of chromosome copy number of disomic or trisomic samples was assessed.

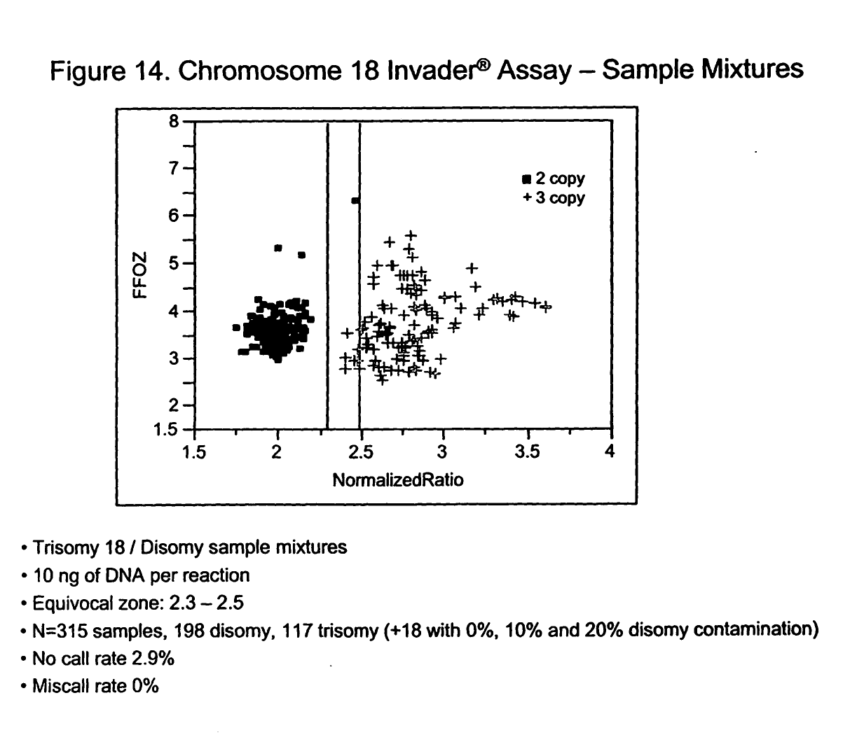

FIG. 14 shows the results from the chromosome 18 Invader assay using gDNA samples of mixed content. The chromosome 18 assay targeted FLJ23403 (SEQ ID NOs: 47 and 146) and CN2 (SEQ ID NOs: 74 and 173). The internal control genes used in this example were ACTA1 (SEQ ID NOs: 1 and 100); and HIST2HBE (SEQ ID NOs: 10 and 109). Reactions were set up and carried out as described in Example 4 section A. In this example, trisomy 18 gDNA samples were mixed with 0, 10, and 20% disomy gDNA, e.g. a 20% disomy contaminated sample (100 ul) contained 20 ul of 2 ng/ul disomy gDNA and 80 ul of 2 ng/ul trisomy gDNA. 5 ul of the mixed content gDNA sample (long total) was added to the Invader assay. Samples that generated normalized ratio values greater than 2.5 were called 3 copy, samples with normalized ratio values between 1.6 and 2.3 were called 2 copy. Samples with normalized ratio values between 2.3 and 2.5 were equivocal (no call samples). These results indicate that the presence of trisomic DNA can be detected in a significant background of disomic or “contaminating” DNA.

The methods described for FIG. 14 were used to evaluate the assay performance for the other gene targets for chromosomes 13, 21, X and Y listed in FIG. 12. Aneuploid samples were mixed with 10, 20 and 30% of a normal sample to mimic varying degrees of maternal cell contamination. Results from these experiments indicate that the Invader assays can detect numerical abnormalities for Chromosomes 13, 18, 21, X and Y with 99% or greater accuracy in the sample mixtures tested.

### Example 5

### Analysis of Triploidy Samples (69, XXY)

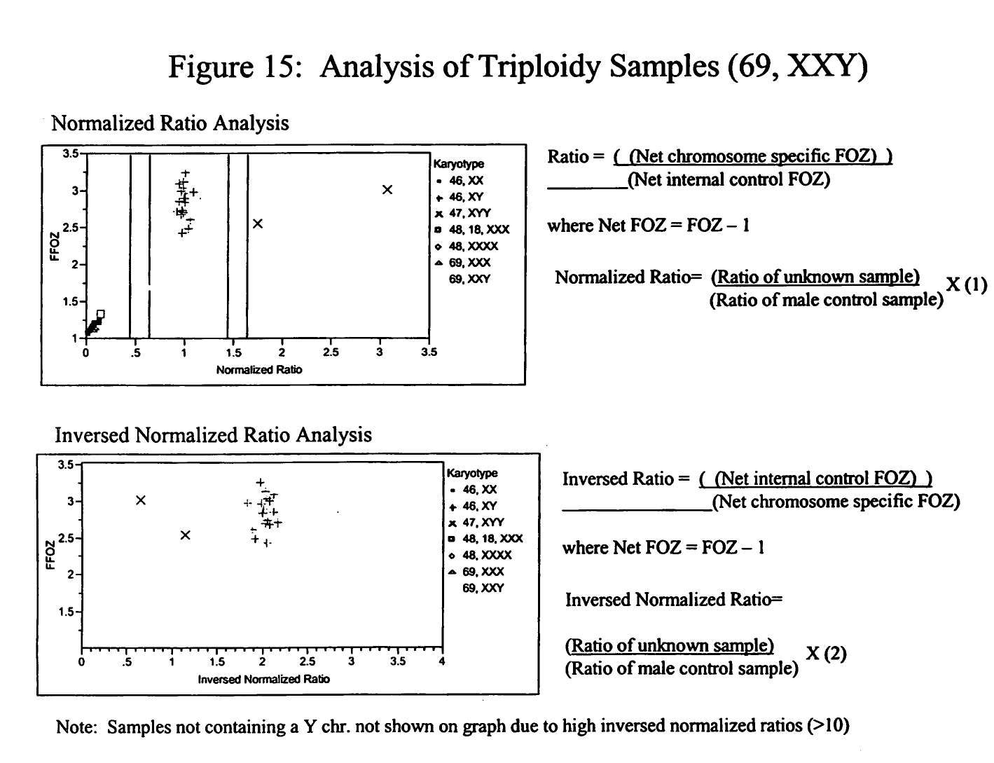

The results of the SRY (Yp11.31) assay (SEQ ID NOs: 33 and 132) biplexed with the alpha-actin internal control are presented in FIG. 15. In this example, 40 genomic DNA samples (25 ng/r×n) were tested including various samples obtained from individuals presumed to be normal males or females (46, XY or 46, XX), as well as various aneuploidy cell line samples obtained from Coriell including (48, +18, XXX; GM03623 48, XXXX; GM01416E and (47, XYY GM01250A and GM09326) and four different triploidy cell lines (69, XXX; GM07744, GM10013 or 69, XXY; AG05025, AG06266).

Normalized ratios for the SRY assay are plotted along the X axis, while the FFOZ for each assay are plotted along the Y axis. Samples that had a RFOZ of at least 1.4 were considered valid. The Normalized Ratio for each sample was generated by dividing the Ratio of each sample by the Ratio of a presumed diploidy male control (obtained from Novagen, cat #70572) and multiplying by 1. Samples were determined to contain 0, 1 or 2 copies of chr. Y based on preliminary estimations for potential copy ranges. Using this method of analysis, the Normalized Ratios for the 69, XXY samples fall within the proposed range for 1 copy Y samples, although the 69, XXY samples do not cluster with the presumed normal male population (46, XY). In order to further distinguish the 69, XXY samples from the 46, XY population, an inverse ratio calculation can be used (divide the alpha actin RFOZ by the chr. Y FFOZ to generate the ratio) and calculate the normalized ratio using the inverse ratios (inverse ratio of unknown sample divided by the inverse ratio of the presumed normal male control sample multiplied by 2. These results demonstrate that using inverse ratios for the chr. Y assay to generate the normalized ratios may be used to determine whether or not a sample has the karyotype of 69, XXY.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method for selecting a chromosome-specific oligonucleotide sequence, comprising:
a. identifying a chromosome-specific genic sequence that is unique in a genome;
b. identifying an exon tag sequence within said genic sequence, wherein said exon tag sequence is compared to said genome to determine that said exon tag sequence is unique within said genome, and
c. selecting an oligonucleotide sequence complementary to said exon tag or its complement.

2. The method of claim 1, wherein said exon tag sequence within said genic sequence is less than 100 base pairs in length.

3. The method of claim 2, wherein said exon tag sequence within said genic sequence is 91 base pairs in length.

4. The method of claim 1, wherein said exon tag sequence is the length of an entire exon.

5. The method of claim 1, wherein said selecting an oligonucleotide sequence complementary to said exon tag or its complement comprises selecting an oligonucleotide sequence having 20% to 70% GC content.

6. A method for detecting aneuploidy of a chromosome in a subject, comprising the steps of:
a. selecting an exon tag sequence for said chromosome;
b. providing a non-amplifying oligonucleotide detection assay configured to detect said exon tag sequence or its complement; and
c. detecting said exon tag with said non-amplifying oligonucleotide detection assay.

7. The method of claim 6, wherein said selecting an exon tag sequence comprises the steps of:
a. identifying a genic sequence that is specific to said chromosome in said subject, and that is unique in the genome of the species of said subject;
b. identifying an exon tag sequence within said genic sequence, wherein said exon tag sequence is compared to said genome to determine that said exon tag sequence is unique within said genome of the species of said subject.

8. The method of claim 6, further comprising providing an internal control and a non-amplifying oligonucleotide detection assay configured to detect said internal control, wherein said internal control target is detected using said non-amplifying oligonucleotide detection assay configured to detect said internal control.

9. A method for detecting aneuploidy of a chromosome in a subject, comprising the steps of:
a. selecting an exon tag sequence for said chromosome;
b. providing a non-amplified oligonucleotide detection assay configured to detect said exon tag sequence or its complement; and
c. detecting said exon tag with said non-amplified oligonucleotide detection assay.

10. The method of claim 9, wherein said selecting an exon tag sequence comprises the steps of:
a. identifying a genic sequence that is specific to said chromosome in said subject, and that is unique in the genome of the species of said subject;
b. identifying an exon tag sequence within said genic sequence, wherein said exon tag sequence is compared to said genome to determine that said exon tag sequence is unique within said genome of the species of said subject.

11. The method of claim 9, further comprising providing an internal control, and a non-amplifying oligonucleotide detection assay configured to detect said internal control, wherein said internal control target is detected using said non-amplifying oligonucleotide detection assay configured to detect said internal control.

12. The method of claim 8 or claim 11, wherein said internal control comprises a sequence from a gene on chromosome 1.

13. The method of claim 6 or claim 9, wherein said chromosome in a subject is selected from the group consisting of chromosomes 13, 18, 21, X and Y.

14. The method of claim 6 or claim 9 wherein said exon tag sequence is contained in a sample type selected from the group consisting of amniocyte cells, cystic hygroma fluid, amniocyte cell culture, amniotic fluid, chorionic villi, fetal urine, fetal skin, and fetal blood.

15. The method of claim 14 wherein maternal nucleic acid is present as a contaminant in said sample.

16. The method of claim 15 wherein maternal DNA comprises < about 80% of the total DNA isolated from said amniocyte cell culture.

17. A kit comprising a non-amplified oligonucleotide detection assay configured for detecting at least one exon tag.

18. The kit of claim 17, wherein said non-amplified oligonucleotide detection assay comprises first and second oligonucleotides configured to form an invasive cleavage structure in combination with a target sequence comprising said at least one exon tag.

19. The kit of claim 18, wherein said first oligonucleotide comprises a 5′ portion and a 3′ portion, wherein said 3′ portion is configured to hybridize to said target sequence, and wherein said 5′ portion is configured to not hybridize to said target sequence.

20. The kit of claim 18, wherein said second oligonucleotide comprises a 5′ portion and a 3′ portion, wherein said 5′ portion is configured to hybridize to said target sequence, and wherein said 3′ portion is configured to not hybridize to said target sequence.

21. The kit of claim 17, wherein said exon tag is from a gene selected from the group consisting of DSCR9, DLEU1, FLJ23403, PFKFB1, NRIP1, SRY, PCDH9, CN2, PRKY, HLCS, MTMR8, FLJ21174, and PCTK1.

22. The kit of claim 17, further comprising an internal control.

23. The kit of claim 22, wherein said internal control comprises a sequence from a gene on chromosome 1.
